(19) United States
(12) Patent Application Publication
(10) Pub. No.: US 2014/0296223 A1
(43) Pub. Date: Oct. 2, 2014

(54) METHOD FOR REGULATING SKIN PIGMENTATION

(71) Applicants: David E. Fisher, Newton, MA (US); Rizwan Haq, Boston, MA (US); Hans Ragnar Valdemar Widlund, Brookline, MA (US)

(72) Inventors: David E. Fisher, Newton, MA (US); Rizwan Haq, Boston, MA (US); Hans Ragnar Valdemar Widlund, Brookline, MA (US)

(73) Assignee: THE GENERAL HOSPITAL CORPORATION, Boston, MA (US)

(21) Appl. No.: 14/342,084

(22) PCT Filed: Aug. 31, 2012
(86) PCT No.: PCT/US2012/053317

§ 371 Date: Jun. 18, 2014

Related U.S. Application Data
(60) Provisional application No. 61/530,197, filed on Sep. 1, 2011.

(51) Int. Cl. A61K 31/13; A61K 31/277; A61K 31/166; C12Q 1/68; A61K 31/357; A61K 31/428; A61K 31/5415; A61K 31/549; A61K 31/4709; A61K 31/404

(52) U.S. Cl. CPC A61K 31/13; A61K 31/4709; A61K 31/277; A61K 31/166; A61K 31/404; A61K 31/357; A61K 31/428; A61K 31/5415; A61K 31/549; C12Q 1/6897
USPC 514/222.5; 564/456; 514/659; 546/177; 514/314; 558/384; 514/519; 564/179; 514/622; 548/492; 514/419; 549/362; 514/452; 548/180; 514/367; 544/39; 514/226.2; 544/8; 435/6.13; 506/10

(57) ABSTRACT

This invention relates to a method for regulating skin pigmentation of a subject, comprising the step of administering the agent for inhibiting the expression or activation of Microphthalmia Transcription Factor (MITF) to the subject who is in need of regulation of skin pigmentation.

### CROSS REFERENCE TO RELATED APPLICATIONS

This application claims benefit under 35 U.S.C. §119(e) of U.S. Provisional Patent Application Ser. No. 61/530,197 filed Sep. 1, 2011.

### FIELD OF THE INVENTION

This invention relates to a method for regulating skin pigmentation of a subject, comprising the step of administering the agent for inhibiting the expression or activation of Microphthalmia Transcription Factor (MITF) to the subject who is in need of regulation of skin pigmentation.

### BACKGROUND OF THE INVENTION

Skin pigmentation is one of the most obvious and aesthetically notable phenotypic properties in human. The skin pigmentation is derived from the presence of melanin pigments within the epidermis-pigments which vary in chemical structure from yellow/red pheomelanins to brown/black eumelanins. The extreme light-skinned phenotype is most commonly observed within “redhead-fairskinned” people, who have been shown to harbor specific gene polymorphisms. The inventors' extensive study of this pigmentation phenotype has led to a significant interest in understanding and manipulating skin pigmentation for multiple potential uses in human.

Control of pigmentation is mediated by an intricate set of intracellular signals within the epidermis. While the enzymatic machinery for conversion of tyrosine into melanin resides within melanocytes, many of the key initiating signals for pigmentation are derived from the overlying keratinocyte population. Similarly, keratinocytes play a crucial role in late steps of pigmentation, because keratinocytes are the cells in which most epidermal melanin eventually resides, due to the exocytic transport of melanosomes from melanocytes.

Within the melanocyte, a key pathway which controls quantity and quality of pigment production is the pathway regulated by Melanocyte Stimulating Hormone (MSH). MSH is a 12 amino acid peptide cleavage product of the propeptide precursor ProOpioMelanoCortin (POMC). MSH binds to a receptor on the surface of melanocytes (Melanocortin 1 Receptor) which is a G-protein coupled receptor that activates adenylate cyclase to induce cAMP production. In turn, elevated cAMP levels result in phosphorylation of the CREB transcription factor, which stimulates transcription of Microphthalmia Transcription Factor (MITF) gene (Lin J Y, Fisher D E: Melanocyte biology and skin pigmentation, Nature 2007, 445:843-850).

MITF is a melanocyte-specific transcription factor, which has been found to control expression of a large number of genes regulating production of melanin. From the discovery as a transcriptional regulator of pigment enzyme genes (Hemesath T J, Steingrimsson E, McGill G, Hansen M J, Vaught J, Hodgkinson C A, Arnheiter H, Copeland N G, Jenkins N A, Fisher D E: microphthalmia, a critical factor in melanocyte development, defines a discrete transcription factor family, Genes Dev 1994, 8:2770-2780), the inventors have found a great deal about multiple pathways which control MITF expression and transcriptional activity. Mutations in MITF can produce complete depigmentation or altered color of pigment (Lin J Y, Fisher D E: Melanocyte biology and skin pigmentation, Nature 2007, 445:843-850). The MITF promoter is regulated by multiple factors which have independently been shown to play key roles in human pigmentation. These include the Wnt pathway (Widlund H R, Horstmann M A, Price E R, Cui J, Lessnick S L, Wu M, He X, Fisher D E: Beta-catenin-induced melanoma growth requires the downstream target Microphthalmia-associated transcription factor., J Cell Biol 2002, 158:1079-1087. Epub 202 September 1016), SOX10 (Huber W E, Price E R, Widlund H R, Du J, Davis I J, Wegner M, Fisher D E: A tissue-restricted cAMP transcriptional response: SOX10 modulates alpha-melanocyte-stimulating hormone-triggered expression of microphthalmia-associated transcription factor in melanocytes., J Biol Chem 2003, 278:45224-45230. Epub 4203 August 45227), and PAX3 (Price E R, Fisher D E: Sensorineural deafness and pigmentation genes: melanocytes and the Mitf transcriptional network, Neuron 2001, 30:15-18). Mutations within MITF, SOX10, and PAX3 occur in patients with Waardenburg Syndrome, with the MITF mutations arising in patients exhibiting the “pure” melanocytic phenotype, whereas SOX10 or PAX3 mutations cause melanocyte defects plus additional abnormalities (Price E R, Fisher D E: Sensorineural deafness and pigmentation genes: melanocytes and the Mitf transcriptional network., Neuron 2001, 30:15-18). Post-translational regulation of MITF occurs via phosphorylation (Hemesath T J, Price E R, Takemoto C, Badalian T, Fisher D E: MAP kinase links the transcription factor Microphthalmia to c-Kit signalling in melanocytes, Nature 1998, 391:298-301), ubiquitination (Wu M, Hemesath T J, Takemoto C M, Horstmann M A, Wells A G, Price E R, Fisher D Z, Fisher D E: c-Kit triggers dual phosphorylations, which couple activation and degradation of the essential melanocyte factor Mi, Genes Dev 2000, 14:301-312), or sumoylation (Miller A J, Levy C, Davis I J, Razin E, Fisher D E: Sumoylation of MITF and its related family members TFE3 and TFEB, J Biol Chem 2005, 280:146-155) of MITF. The functional consequences of these modifications are partially understood at this time, though additional details remain to be understood. Collectively, the multiple levels of control of MITF expression and activity represent potential opportunities for small molecule modulation of pigmentation.

### SUMMARY OF THE INVENTION

Control of pigmentation has been shown to occur largely through regulation of the MITF transcription factor within melanocytes. Multiple signaling pathways converge on this factor, including the Melanocyte Stimulating Hormone (MSH), Endothelin, c-Kit, and c-MET pathways within melanocytes. The sources of these signals largely arise from adjacent keratinocytes within the epidermis, and ultraviolet radiation is an important trigger of some of these stimulatory factors. Hyperpigmentation is a common and cosmetically troubling problem within many human populations. Its causes are incompletely understood, but include hormonal, post-inflammatory, age-related, and other etiologies. Current strategies for treatment of hyperpigmentation are largely limited to inhibition of the tyrosinase enzyme, a strategy which shows limited efficacy and some associated toxicity.

One aspect of the invention provides a method for regulating skin pigmentation of a subject. In some embodiments, the method for regulating skin pigmentation of a subject, comprises the step of administering an agent for inhibiting the expression or activation of Microphthalmia Transcription Factor (MITF) to the subject who is in need of regulation of skin pigmentation. In some embodiments, the agent for inhibiting the expression or activation of MITF comprises a compound identified using the small molecule screen of the invention. In some embodiments, the agent for inhibiting the expression or activation of MITF can be selected from the group consisting of:
1-[(1S,3R,5R,7S)-3-(3,4-dimethylphenyl)adamantan-1-yl]methanamine;
5-chloro-7-[(2-chloro-6-fluorophenyl)(pyridin-2-ylamino)methyl]quinolin-8-ol;
(1S,2R,3S,6S)-1-benzoyl-6-tert-butylspiro[2.5]octane-1,2-dicarbonitrile;
N-[(4-chlorophenyl)(2-hydroxy-1-naphthyl)methyl]-3-methoxybenzamide;
Ethyl-1-benzyl-5-[3-(tert-butylamino)-2-hydroxypropoxy]-2-methyl-1H-indole-3-carboxylate;
5-chloro-7-[(2,5-dimethylphenyl)(pyridin-2-ylamino)methyl]quinolin-8-ol;
N-[5-(2,3-dihydro-1,4-benzodioxin-6-yl)-2-methyl-3-furoyl]glycine;
[3-(1,3-benzothiazol-2-yl)phenoxy]acetic acid;
N-(2-fluorophenyl)-2-{[5-(3-methylphenyl)-1,3,4-oxadiazol-2-yl]thio}acetamide;
N-[4-(6-methyl-1,3-benzothiazol-2-yl)phenyl]-2-[(5-methyl-1,3,4-thiadiazol-2-yl)thio]acetamide;
2-(2-bromo-4-tert-butylphenoxy)-N-(2-methoxy-5-nitrophenyl)acetamide;
Methyl-5-{[(4-fluoro-2-methylphenyl)sulfonyl](isonicotinoyl)amino}-2-methyl-1-benzofuran-3-carboxylate;
1-(4-chlorobenzyl)-N-(pyridin-3-ylmethyl)-1H-pyrazolo[3,4-d]pyrimidin-4-amine;
2-{[5-(4-chlorophenyl)-4-(3-methylphenyl)-4H-1,2,4-triazol-3-yl]thio}-N-(3-methoxyphenyl)acetamide;
N-phenyl-3-[(4-phenyl-1,3-thiazol-2-yl)thio]propanamide;
Ethyl-[3-(1,3-benzothiazol-2-yl)phenoxy]acetate;
2-methyl-3-[(4-methylpiperidin-1-yl)carbonyl]naphtho[2,3-b]furan-4,9-dione;
N-(3-phenylpropyl)-10H-phenothiazine-10-carboxamide;
5-(4-chlorophenyl)-2-pyrrolidin-1-yl-6H-1,3,4-thiadiazine;
7-(4-fluorophenyl)-1-(2-furylmethyl)-5,5-bis(trifluoromethyl)-5,8-dihydropyrimido[4,5-d]pyrimidine-2,4(1H,3H)-dione;
3-{[(2-bromo-4-methylphenoxy)acetyl]amino}benzoic acid;
3-{[4-(hydroxymethyl)-2-methoxyphenoxy]methyl}benzoic acid;
5-phenyl[1,2,5]oxadiazolo[3,4-b]pyrazine;
2,3,9-trimethoxy-6a,12a-dihydrochromoeo[3,4-b]chromen-12(6H)-one;
4-[(4-chlorobenzyl)oxy]-3-methoxy-N-(2-methyl-5-[1,3]oxazolo[4,5-b]pyridin-2-ylphenyl)benzamide;
3-(1,3-benzothiazol-2-ylthio)propanoic acid;
3-{[(4-ethoxyphenoxy)acetyl]amino}benzoic acid;
N-[4-chloro-3-(trifluoromethyl)phenyl]-1-[(2,5-dimethoxyphenyl)sulfonyl]piperidine-4-carboxamide;
N-[3,5-bis(trifluoromethyl)phenyl]-2-[(5-nitro-1H-benzimidazol-2-yl)thio]acetamide;
5-[4-(4-tert-butylbenzoyl)piperazin-1-yl]-2-nitro-N-(pyridin-3-ylmethyl)aniline;
3-{[2-chloro-4-(hydroxymethyl)phenoxy]methyl}benzoic acid;
{[(2-chlorophenyl)amino][(3-fluorobenzyl)thio]methylene}malononitrile;
{[(4-bromophenyl)amino][(4-fluorobenzyl)thio]methylene}malononitrile; and
1-[(1S,3R,5R,7S)-3-(3,4-dimethylphenyl)adamantan-1-yl]methanamine;
(3,5-di-t-butyl-4-hydroxybenzylidene)malononitrile;
2-amino-N-{5-[6-(dimethylamino)-9H-purin-9-yl]-4-hydroxy-2-(hydroxymethyl)tetrahydrofuran-3-yl}-3-(4-methoxyphenyl)propanamide;
1,3,6-trihydroxy-7-methoxy-2,8-bis(3-methylbut-2-en-1-yl)-9H-xanthen-9-one;
(3S,3aR,4R,6S,6aR,7S,8R,9bR)-6-acetoxy-4-(butyryloxy)-3,3a-dihydroxy-3,6,9-trimethyl-8-{[(2Z)-2-methylbut-2-enoyl]oxy}-2-oxo-2,3,3a,4,5,6,6a,7,8,9b-decahydroazuleno[4,5-b]furan-7-ylhexanoate;
(1E,5S)-6,6,6-trifluoro-5-hydroxy-1,5-diphenylhex-1-en-3-one; 2-(4-methoxyphenyl)-1,3-benzothiazole;
Ethyl-4-amino-2-{[(1E)-3-(4-methylphenyl)-3-oxoprop-1-en-1-yl]thio}pyrimidine-5-carboxylate;
3-[(5-chloro-1,3-benzothiazol-2-yl)thio]-5,5-dimethylcyclohex-2-en-1-one;
(2E)-3-[(5-methyl-1H-benzimidazol-2-yl)thio]-1-phenylprop-2-en-1-one;
11,12-dihydroxy-3-isobutyl-4,5,8-trimethyl-3,3a,4,6a,9,10,11,12-octahydro-1H-cycloundeca[d]isoindole-1,15(2H)-dione;
3-(hydroxymethyl)-6-isopropyl-5-[(2-methylbut-2-enoyl)oxy]-2-oxocyclohex-3-en-1-yl4-hydroxy-2-methylbut-2-enoate;
1,11,12-trihydroxy-2,16-dioxo-11,20-epoxypicras-3-en-15-yl2-hydroxy-2-methylbutanoate;
1-[3,5-bis(trifluoromethyl)phenyl]-3-[3-chloro-4-(isopropylsulfonyl)-2-thienyl]urea;

a pharmaceutically acceptable salt thereof;

and any combination thereof.

In some embodiments, the agent for inhibiting the expression or activation of MITF can be selected from the group consisting of:
1-[(1S,3R,5R,7S)-3-(3,4-dimethylphenyl)adamantan-1-yl]methanamine;
5-chloro-7-[(2-chloro-6-fluorophenyl)(pyridin-2-ylamino)methyl]quinolin-8-ol;
(1S,2R,3S,6S)-1-benzoyl-6-tert-butylspiro[2.5]octane-1,2-dicarbonitrile;
N-[(4-chlorophenyl)(2-hydroxy-1-naphthyl)methyl]-3-methoxybenzamide;
Ethyl-1-benzyl-5-[3-(tert-butylamino)-2-hydroxypropoxy]-2-methyl-1H-indole-3-carboxylate;
5-chloro-7-[(2,5-dimethylphenyl)(pyridin-2-ylamino)methyl]quinolin-8-ol;
N-[5-(2,3-dihydro-1,4-benzodioxin-6-yl)-2-methyl-3-furoyl]glycine;
[3-(1,3-benzothiazol-2-yl)phenoxy]acetic acid;
N-(2-fluorophenyl)-2-{[5-(3-methylphenyl)-1,3,4-oxadiazol-2-yl]thio}acetamide;
N-[4-(6-methyl-1,3-benzothiazol-2-yl)phenyl]-2-[(5-methyl-1,3,4-thiadiazol-2-yl)thio]acetamide;
2-(2-bromo-4-tert-butylphenoxy)-N-(2-methoxy-5-nitrophenyl)acetamide;
Methyl-5-{[(4-fluoro-2-methylphenyl)sulfonyl](isonicotinoyl)amino}-2-methyl-1-benzofuran-3-carboxylate;
1-(4-chlorobenzyl)-N-(pyridin-3-ylmethyl)-1H-pyrazolo[3,4-d]pyrimidin-4-amine;
2-{[5-(4-chlorophenyl)-4-(3-methylphenyl)-4H-1,2,4-triazol-3-yl]thio}-N-(3-methoxyphenyl)acetamide;
N-phenyl-3-[(4-phenyl-1,3-thiazol-2-yl)thio]propanamide; Ethyl-[3-(1,3-benzothiazol-2-yl)phenoxy]acetate;
2-methyl-3-[(4-methylpiperidin-1-yl)carbonyl]naphtho[2,3-b]furan-4,9-dione;
N-(3-phenylpropyl)-10H-phenothiazine-10-carboxamide;
5-(4-chlorophenyl)-2-pyrrolidin-1-yl-6H-1,3,4-thiadiazine;
7-(4-fluorophenyl)-1-(2-furylmethyl)-5,5-bis(trifluoromethyl)-5,8-dihydropyrimido[4,5-d]pyrimidine-2,4(1H,3H)-dione;
3-{[(2-bromo-4-methylphenoxy)acetyl]amino}benzoic acid;
3-{[4-(hydroxymethyl)-2-methoxyphenoxy]methyl}benzoic acid;
5-phenyl[1,2,5]oxadiazolo[3,4-b]pyrazine;
2,3,9-trimethoxy-6a,12a-dihydrochromoeo[3,4-b]chromen-12(6H)-one;
4-[(4-chlorobenzyl)oxy]-3-methoxy-N-(2-methyl-5-[1,3]oxazolo[4,5-b]pyridin-2-ylphenyl)benzamide;
3-(1,3-benzothiazol-2-ylthio)propanoic acid;
3-{[(4-ethoxyphenoxy)acetyl]amino}benzoic acid;
N-[4-chloro-3-(trifluoromethyl)phenyl]-1-[(2,5-dimethoxyphenyl)sulfonyl]piperidine-4-carboxamide;
N-[3,5-bis(trifluoromethyl)phenyl]-2-[(5-nitro-1H-benzimidazol-2-yl)thio]acetamide;
5-[4-(4-tert-butylbenzoyl)piperazin-1-yl]-2-nitro-N-(pyridin-3-ylmethyl)aniline;
3-{[2-chloro-4-(hydroxymethyl)phenoxy]methyl}benzoic acid;
{[(2-chlorophenyl)amino][(3-fluorobenzyl)thio]methylene}malononitrile;
{[(4-bromophenyl)amino][(4-fluorobenzyl)thio]methylene}malononitrile;
1-[(1S,3R,5R,7S)-3-(3,4-dimethylphenyl)adamantan-1-yl]methanamine;

a pharmaceutically acceptable thereof;

and any combination thereof.

In some embodiments, the agent for inhibiting the expression or activation of MITF can be selected from the group consisting of:
(3,5-di-t-butyl-4-hydroxybenzylidene)malononitrile;
2-amino-N-{5-[6-(dimethylamino)-9H-purin-9-yl]-4-hydroxy-2-(hydroxymethyl)tetrahydrofuran-3-yl}-3-(4-methoxyphenyl)propanamide;
1,3,6-trihydroxy-7-methoxy-2,8-bis(3-methylbut-2-en-1-yl)-9H-xanthen-9-one;
(3S,3aR,4R,6S,6aR,7S,8R,9bR)-6-acetoxy-4-(butyryloxy)-3,3a-dihydroxy-3,6,9-trimethyl-8-{[(2Z)-2-methylbut-2-enoyl]oxy}-2-oxo-2,3,3a,4,5,6,6a,7,8,9b-decahydroazuleno[4,5-b]furan-7-ylhexanoate;
(1E,5S)-6,6,6-trifluoro-5-hydroxy-1,5-diphenylhex-1-en-3-one;
2-(4-methoxyphenyl)-1,3-benzothiazole;
Ethyl-4-amino-2-{[(1E)-3-(4-methylphenyl)-3-oxoprop-1-en-1-yl]thio}pyrimidine-5-carboxylate;
3-[(5-chloro-1,3-benzothiazol-2-yl)thio]-5,5-dimethylcyclohex-2-en-1-one;
(2E)-3-[(5-methyl-1H-benzimidazol-2-yl)thio]-1-phenylprop-2-en-1-one;
11,12-dihydroxy-3-isobutyl-4,5,8-trimethyl-3,3a,4,6a,9,10,11,12-octahydro-1H-cycloundeca[d]isoindole-1,15(2H)-dione;
3-(hydroxymethyl)-6-isopropyl-5-[(2-methylbut-2-enoyl)oxy]-2-oxocyclohex-3-en-1-yl4-hydroxy-2-methylbut-2-enoate;
1,11,12-trihydroxy-2,16-dioxo-11,20-epoxypicras-3-en-15-yl2-hydroxy-2-methylbutanoate;
1-[3,5-bis(trifluoromethyl)phenyl]-3-[3-chloro-4-(isopropylsulfonyl)-2-thienyl]urea;

a pharmaceutically acceptable salt thereof;

and any combination thereof.

In some embodiments, the agent for inhibiting the expression or activation of MITF can be selected from the group consisting of:
1,3,6-trihydroxy-7-methoxy-2,8-bis(3-methylbut-2-en-1-yl)-9H-xanthen-9-one;
(1E,5S)-6,6,6-trifluoro-5-hydroxy-1,5-diphenylhex-1-en-3-one;
3-(hydroxymethyl)-6-isopropyl-5-[(2-methylbut-2-enoyl)oxy]-2-oxocyclohex-3-en-1-yl-4-hydroxy-2-methylbut-2-enoate;

a pharmaceutically acceptable salt thereof;

and any combination thereof.

MITF plays a central role in the differentiation/pigmentation of melanocytes. MITF expression is regulated by the cAMP pathway downstream of Melanocyte Stimulating Hormone (MSH), and transcriptional targets thereof include tyrosinase and most enzymes which actively participate in the production of melanin from tyrosine. Hence, one aspect of the invention comprises identifying compounds having the ability for inhibiting the expression and/or activity of Microphthalmia Transcription Factor (MITF), which can be used for regulating skin pigmentation of a subject, in particular a human.

Another aspect of the invention provides a small molecule screen to identify compounds capable of downregulating the activity of the MITF transcription factor

### DETAILED DESCRIPTION OF THE INVENTION

One aspect of the invention provides a method for regulating skin pigmentation of a subject. One way of regulating skin pigmentation is by controlling melanocytes and inhibiting hyperpigmentation. Regulation of skin pigmentation, is at issue in numerous conditions such as Acanthosis Nigricans, albinism, hyperpigmentation, incontinentia pigmenti, liver spots and aging hands, mcCune-Albright syndrome, melisma, progressive pigmentary purpura, Sturge-Weber Syndrome, Xeroderma pigmentosum, and Waardenburg Syndrome. Control of pigmentation has been shown to occur largely through regulation of the MITF transcription factor within melanocytes. Multiple signaling pathways converge on this factor, including the Melanocyte Stimulating Hormone (MSH), Endothelin, c-Kit, and c-MET pathways within melanocytes. The sources of these signals largely arise from adjacent keratinocytes within the epidermis, and ultraviolet radiation is an important trigger of some of these stimulatory factors.

In some embodiments, the method for regulating skin pigmentation of a subject, comprises the step of administering an agent for inhibiting the expression or activation of Microphthalmia Transcription Factor (MITF) to the subject who is in need of regulation of skin pigmentation.

In order to identify agents capable of inhibiting the expression or activation of MITF, a screening assay system was developed. In come embodiments, the compound described herein can inhibit the expression of MITF by at least 5% (e.g. 5%, 10%, 15%, 20%, 25%, 30%, 35%, 40%, 45%, 50%, 55%, 60%, 65%, 70%, 75%, 80%, 85%, 90, 95%, 99%, 100%) relative to a control or reference level. In come embodiments, the compound described herein can inhibit the activation of MITF by at least 5% (e.g. 5%, 10%, 15%, 20%, 25%, 30%, 35%, 40%, 45%, 50%, 55%, 60%, 65%, 70%, 75%, 80%, 85%, 90, 95%, 99%, 100%) relative to a control or reference level. It is to be understood that complete inhibition of MITF expression or activation is not required.

In accordance with the screening assay system, inventors have identified compounds having the ability to inhibit the expression or activation of Microphthalmia Transcription Factor (MITF).

In some embodiments, the compounds having the ability of inhibiting the expression or activation of MITF are selected from the group consisting of:
1-[(1S,3R,5R,7S)-3-(3,4-dimethylphenyl)adamantan-1-yl]methanamine;
5-chloro-7-[(2-chloro-6-fluorophenyl)(pyridin-2-ylamino)methyl]quinolin-8-ol;
(1S,2R,3S,6S)-1-benzoyl-6-tert-butylspiro[2.5]octane-1,2-dicarbonitrile;
N-[(4-chlorophenyl)(2-hydroxy-1-naphthyl)methyl]-3-methoxybenzamide;
Ethyl-1-benzyl-5-[3-(tert-butylamino)-2-hydroxypropoxy]-2-methyl-1H-indole-3-carboxylate;
5-chloro-7-[(2,5-dimethylphenyl)(pyridin-2-ylamino)methyl]quinolin-8-ol;
N-[5-(2,3-dihydro-1,4-benzodioxin-6-yl)-2-methyl-3-furoyl]glycine; [3-(1,3-benzothiazol-2-yl)phenoxy]acetic acid;
N-(2-fluorophenyl)-2-{[5-(3-methylphenyl)-1,3,4-oxadiazol-2-yl]thio}acetamide;
N-[4-(6-methyl-1,3-benzothiazol-2-yl)phenyl]-2-[(5-methyl-1,3,4-thiadiazol-2-yl)thio]acetamide;
2-(2-bromo-4-tert-butylphenoxy)-N-(2-methoxy-5-nitrophenyl)acetamide;
Methyl-5-{[(4-fluoro-2-methylphenyl)sulfonyl](isonicotinoyl)amino}-2-methyl-1-benzofuran-3-carboxylate;
1-(4-chlorobenzyl)-N-(pyridin-3-ylmethyl)-1H-pyrazolo[3,4-d]pyrimidin-4-amine;
2-{[5-(4-chlorophenyl)-4-(3-methylphenyl)-4H-1,2,4-triazol-3-yl]thio}-N-(3-methoxyphenyl)acetamide;
N-phenyl-3-[(4-phenyl-1,3-thiazol-2-yl)thio]propanamide;
Ethyl-[3-(1,3-benzothiazol-2-yl)phenoxy]acetate;
2-methyl-3-[(4-methylpiperidin-1-yl)carbonyl]naphtho[2,3-b]furan-4,9-dione;
N-(3-phenylpropyl)-10H-phenothiazine-10-carboxamide;
5-(4-chlorophenyl)-2-pyrrolidin-1-yl-6H-1,3,4-thiadiazine;
7-(4-fluorophenyl)-1-(2-furylmethyl)-5,5-bis(trifluoromethyl)-5,8-dihydropyrimido[4,5-d]pyrimidine-2,4(1H,3H)-dione;
3-{[(2-bromo-4-methylphenoxy)acetyl]amino}benzoic acid;
3-{[4-(hydroxymethyl)-2-methoxyphenoxy]methyl}benzoic acid;
5-phenyl[1,2,5]oxadiazolo[3,4-b]pyrazine;
2,3,9-trimethoxy-6a,12a-dihydrochromoeo[3,4-b]chromen-12(6H)-one;
4-[(4-chlorobenzyl)oxy]-3-methoxy-N-(2-methyl-5-[1,3]oxazolo[4,5-b]pyridin-2-ylphenyl)benzamide;
3-(1,3-benzothiazol-2-ylthio)propanoic acid;
3-{[(4-ethoxyphenoxy)acetyl]amino}benzoic acid;
N-[4-chloro-3-(trifluoromethyl)phenyl]-1-[(2,5-dimethoxyphenyl)sulfonyl]piperidine-4-carboxamide;
N-[3,5-bis(trifluoromethyl)phenyl]-2-[(5-nitro-1H-benzimidazol-2-yl)thio]acetamide;
5-[4-(4-tert-butylbenzoyl)piperazin-1-yl]-2-nitro-N-(pyridin-3-ylmethyl)aniline;
3-{[2-chloro-4-(hydroxymethyl)phenoxy]methyl}benzoic acid;
{[(2-chlorophenyl)amino][(3-fluorobenzyl)thio]methylene}malononitrile; {[(4-bromophenyl)amino][(4-fluorobenzyl)thio]methylene}malononitrile; and
1-[(1S,3R,5R,7S)-3-(3,4-dimethylphenyl)adamantan-1-yl]methanamine;
(3,5-di-t-butyl-4-hydroxybenzylidene)malononitrile;
2-amino-N-{5-[6-(dimethylamino)-9H-purin-9-yl]-4-hydroxy-2-(hydroxymethyl)tetrahydrofuran-3-yl}-3-(4-methoxyphenyl)propanamide;
1,3,6-trihydroxy-7-methoxy-2,8-bis(3-methylbut-2-en-1-yl)-9H-xanthen-9-one;
(3S,3aR,4R,6S,6aR,7S,8R,9bR)-6-acetoxy-4-(butyryloxy)-3,3a-dihydroxy-3,6,9-trimethyl-8-{[(2Z)-2-methylbut-2-enoyl]oxy}-2-oxo-2,3,3a,4,5,6,6a,7,8,9b-decahydroazuleno[4,5-b]furan-7-ylhexanoate;
(1E,5S)-6,6,6-trifluoro-5-hydroxy-1,5-diphenylhex-1-en-3-one; 2-(4-methoxyphenyl)-1,3-benzothiazole;
Ethyl-4-amino-2-{[(1E)-3-(4-methylphenyl)-3-oxoprop-1-en-1-yl]thio}pyrimidine-5-carboxylate;
3-[(5-chloro-1,3-benzothiazol-2-yl)thio]-5,5-dimethylcyclohex-2-en-1-one;
(2E)-3-[(5-methyl-1H-benzimidazol-2-yl)thio]-1-phenylprop-2-en-1-one;
11,12-dihydroxy-3-isobutyl-4,5,8-trimethyl-3,3a,4,6a,9,10,11,12-octahydro-1H-cycloundeca[d]isoindole-1,15(2H)-dione;
3-(hydroxymethyl)-6-isopropyl-5-[(2-methylbut-2-enoyl)oxy]-2-oxocyclohex-3-en-1-yl4-hydroxy-2-methylbut-2-enoate;
1,11,12-trihydroxy-2,16-dioxo-11,20-epoxypicras-3-en-15-yl2-hydroxy-2-methylbutanoate;
1-[3,5-bis(trifluoromethyl)phenyl]-3-[3-chloro-4-(isopropylsulfonyl)-2-thienyl]urea;

a pharmaceutically acceptable salt thereof;

and any combination thereof.

In some embodiments, the compounds having the ability of inhibiting the expression or activation of MITF are selected from the group consisting of:
1-[(1S,3R,5R,7S)-3-(3,4-dimethylphenyl)adamantan-1-yl]methanamine;
5-chloro-7-[(2-chloro-6-fluorophenyl)(pyridin-2-ylamino)methyl]quinolin-8-ol;
(1S,2R,3S,6S)-1-benzoyl-6-tert-butylspiro[2.5]octane-1,2-dicarbonitrile;
N-[(4-chlorophenyl)(2-hydroxy-1-naphthyl)methyl]-3-methoxybenzamide;
Ethyl-1-benzyl-5-[3-(tert-butylamino)-2-hydroxypropoxy]-2-methyl-1H-indole-3-carboxylate;
5-chloro-7-[(2,5-dimethylphenyl)(pyridin-2-ylamino)methyl]quinolin-8-ol;
N-[5-(2,3-dihydro-1,4-benzodioxin-6-yl)-2-methyl-3-furoyl]glycine;
[3-(1,3-benzothiazol-2-yl)phenoxy]acetic acid;
N-(2-fluorophenyl)-2-{[5-(3-methylphenyl)-1,3,4-oxadiazol-2-yl]thio}acetamide;
N-[4-(6-methyl-1,3-benzothiazol-2-yl)phenyl]-2-[(5-methyl-1,3,4-thiadiazol-2-yl)thio]acetamide;
2-(2-bromo-4-tert-butylphenoxy)-N-(2-methoxy-5-nitrophenyl)acetamide;
Methyl-5-{[(4-fluoro-2-methylphenyl)sulfonyl](isonicotinoyl)amino}-2-methyl-1-benzofuran-3-carboxylate;
1-(4-chlorobenzyl)-N-(pyridin-3-ylmethyl)-1H-pyrazolo[3,4-d]pyrimidin-4-amine;
2-{[5-(4-chlorophenyl)-4-(3-methylphenyl)-4H-1,2,4-triazol-3-yl]thio}-N-(3-methoxyphenyl)acetamide;
N-phenyl-3-[(4-phenyl-1,3-thiazol-2-yl)thio]propanamide;
Ethyl-[3-(1,3-benzothiazol-2-yl)phenoxy]acetate;
2-methyl-3-[(4-methylpiperidin-1-yl)carbonyl]naphtho[2,3-b]furan-4,9-dione;
N-(3-phenylpropyl)-10H-phenothiazine-10-carboxamide;
5-(4-chlorophenyl)-2-pyrrolidin-1-yl-6H-1,3,4-thiadiazine;
7-(4-fluorophenyl)-1-(2-furylmethyl)-5,5-bis(trifluoromethyl)-5,8-dihydropyrimido[4,5-d]pyrimidine-2,4(1H,3H)-dione;
3-{[(2-bromo-4-methylphenoxy)acetyl]amino}benzoic acid;
3-{[4-(hydroxymethyl)-2-methoxyphenoxy]methyl}benzoic acid;
5-phenyl[1,2,5]oxadiazolo[3,4-b]pyrazine;
2,3,9-trimethoxy-6a,12a-dihydrochromoeo[3,4-b]chromen-12(6H)-one;
4-[(4-chlorobenzyl)oxy]-3-methoxy-N-(2-methyl-5-[1,3]oxazolo[4,5-b]pyridin-2-ylphenyl)benzamide;
3-(1,3-benzothiazol-2-ylthio)propanoic acid;
3-{[(4-ethoxyphenoxy)acetyl]amino}benzoic acid;
N-[4-chloro-3-(trifluoromethyl)phenyl]-1-[(2,5-dimethoxyphenyl)sulfonyl]piperidine-4-carboxamide;
N-[3,5-bis(trifluoromethyl)phenyl]-2-[(5-nitro-1H-benzimidazol-2-yl)thio]acetamide;
5-[4-(4-tert-butylbenzoyl)piperazin-1-yl]-2-nitro-N-(pyridin-3-ylmethyl)aniline;
3-{[2-chloro-4-(hydroxymethyl)phenoxy]methyl}benzoic acid;
{[(2-chlorophenyl)amino][(3-fluorobenzyl)thio]methylene}malononitrile;
{[(4-bromophenyl)amino][(4-fluorobenzyl)thio]methylene}malononitrile; and
1-[(1S,3R,5R,7S)-3-(3,4-dimethylphenyl)adamantan-1-yl]methanamine;

a pharmaceutically acceptable salt thereof;

and any combination thereof.

In some embodiments, the compounds having the ability of inhibiting the expression or activation of MITF are selected from the group consisting of:
(3,5-di-t-butyl-4-hydroxybenzylidene)malononitrile;
2-amino-N-{5-[6-(dimethylamino)-9H-purin-9-yl]-4-hydroxy-2-(hydroxymethyl)tetrahydrofuran-3-yl}-3-(4-methoxyphenyl)propanamide;
1,3,6-trihydroxy-7-methoxy-2,8-bis(3-methylbut-2-en-1-yl)-9H-xanthen-9-one;
(3S,3aR,4R,6S,6aR,7S,8R,9bR)-6-acetoxy-4-(butyryloxy)-3,3a-dihydroxy-3,6,9-trimethyl-8-{[(2Z)-2-methylbut-2-enoyl]oxy}-2-oxo-2,3,3a,4,5,6,6a,7,8,9b-decahydroazuleno[4,5-b]furan-7-ylhexanoate;
(1E,5S)-6,6,6-trifluoro-5-hydroxy-1,5-diphenylhex-1-en-3-one;
2-(4-methoxyphenyl)-1,3-benzothiazole;
Ethyl-4-amino-2-{[(1E)-3-(4-methylphenyl)-3-oxoprop-1-en-1-yl]thio}pyrimidine-5-carboxylate;
3-[(5-chloro-1,3-benzothiazol-2-yl)thio]-5,5-dimethylcyclohex-2-en-1-one;
(2E)-3-[(5-methyl-1H-benzimidazol-2-yl)thio]-1-phenylprop-2-en-1-one;
11,12-dihydroxy-3-isobutyl-4,5,8-trimethyl-3,3a,4,6a,9,10,11,12-octahydro-1H-cycloundeca[d]isoindole-1,15(2H)-dione;
3-(hydroxymethyl)-6-isopropyl-5-[(2-methylbut-2-enoyl)oxy]-2-oxocyclohex-3-en-1-yl4-hydroxy-2-methylbut-2-enoate;
1,11,12-trihydroxy-2,16-dioxo-11,20-epoxypicras-3-en-15-yl2-hydroxy-2-methylbutanoate;
1-[3,5-bis(trifluoromethyl)phenyl]-3-[3-chloro-4-(isopropylsulfonyl)-2-thienyl]urea;

a pharmaceutically acceptable salt thereof;

and any combination thereof.

In some embodiments, the compounds having the ability of inhibiting the expression or activation of MITF are selected from the group consisting of:
1,3,6-trihydroxy-7-methoxy-2,8-bis(3-methylbut-2-en-1-yl)-9H-xanthen-9-one;
(1E,5S)-6,6,6-trifluoro-5-hydroxy-1,5-diphenylhex-1-en-3-one;
3-(hydroxymethyl)-6-isopropyl-5-[(2-methylbut-2-enoyl)oxy]-2-oxocyclohex-3-en-1-yl-4-hydroxy-2-methylbut-2-enoate;

a pharmaceutically acceptable salt thereof;

and any combination thereof.

In some embodiments, the agent for inhibiting the expression or activation of Microphthalmia Transcription Factor (MITF), can be administered parenterally, and the agents can be prepared as, for example, a external composition.

### Pharmaceutical Compositions

In one aspect of the present invention, pharmaceutical compositions are provided, which comprise one or more (e.g. 1, 2, 3, 4, 5, 6, 7, 8, 9, 10, or more) of the compounds described herein (or a prodrug, pharmaceutically acceptable salt, or other pharmaceutically acceptable form thereof), and optionally a pharmaceutically acceptable excipient. In certain embodiments, these compositions optionally further comprise one or more additional therapeutic agents. Alternatively, a compound of the invention can be administered to a patient in need thereof in combination with the administration of one or more other therapeutic agents. For example, in the regulation of skin pigmentation, an additional therapeutic agents for conjoint administration or inclusion in a pharmaceutical composition with a compound of this invention can be an approved skin pigmentation agent.

Amount of the compound or compounds in the pharmaceutical composition can be based on weight, moles, or volume. In some embodiments, the pharmaceutical composition comprises at least 0.0001% compounds of the invention. In some embodiments, the pharmaceutical composition comprises at least 0.1% compounds of the invention. In some embodiments, the pharmaceutical composition comprises at least 0.5% compounds of the invention. In some embodiments, the pharmaceutical composition comprises at least 1% compounds of the invention. In some embodiments, the pharmaceutical composition comprises at least 2% compounds of the invention. In some embodiments, the pharmaceutical composition comprises at least 3% compounds of the invention. In some embodiments, the pharmaceutical composition comprises at least 4% compounds of the invention. In some embodiments, the pharmaceutical composition comprises at least 5% compounds of the invention. In some embodiments, the pharmaceutical composition comprises at least 10% compounds of the invention. In some embodiments, the pharmaceutical composition comprises 0.01%-99% of the compounds of the invention. In some embodiments, the pharmaceutical composition comprises 0.05%-90% of the compounds of the invention. In some embodiments, the pharmaceutical composition comprises 0.1%-85% of the compounds of the invention. In some embodiments, the pharmaceutical composition comprises 0.5%-80% of the compounds of the invention. In some embodiments, the pharmaceutical composition comprises 1%-75% of the compounds of the invention. In some embodiments, the pharmaceutical composition comprises 2%-70% of the compounds of the invention. In some embodiments, the pharmaceutical composition comprises 3%-65% of the compounds of the invention. In some embodiments, the pharmaceutical composition comprises 4%-60% of the compounds of the invention. In some embodiments, the pharmaceutical composition comprises 5%-50% of the compounds of the invention.

It will also be appreciated that certain of the compounds of present invention can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or a pro-drug or other adduct or derivative of a compound of this invention which upon administration to a patient in need is capable of providing, directly or indirectly, a compound as otherwise described herein, or a metabolite or residue thereof.

As described above, the pharmaceutical compositions of the present invention optionally comprise a pharmaceutically acceptable excipient, which, as used herein, includes any and all solvents, diluents, or other liquid vehicle, dispersion or suspension aids, surface active agents, isotonic agents, thickening or emulsifying agents, preservatives, antioxidants, solid binders, lubricants, and the like, as suited to the particular dosage form desired. Remington's Pharmaceutical Sciences, Sixteenth Edition, E. W. Martin (Mack Publishing Co., Easton, Pa., 1980) discloses various excipients used in formulating pharmaceutical compositions and known techniques for the preparation thereof. Except insofar as any conventional excipient medium is incompatible with the compounds of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatine; talc; excipients such as cocoa butter and suppository waxes; oils such as peanut oil, cottonseed oil; safflower oil, sesame oil; olive oil; corn oil, and soybean oil; glycols; such as propylene glycol; esters such as ethyl oleate and ethyl laurate; agar, buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives, and antioxidants can also be present in the composition, according to the judgment of the formulator.

Liquid dosage forms for oral administration include, but are not limited to, pharmaceutically acceptable emulsions, microemulsions, solutions, suspensions, syrups and elixirs. In addition to the active compounds, the liquid dosage forms can contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, com, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions can be formulated according to the known art using suitable dispersing or wetting agents and suspending agents. The sterile injectable preparation can also be a sterile injectable solution, suspension or emulsion in a nontoxic parenterally acceptable diluent or solvent, for example, as a solution in 1,3-butanediol. Among the acceptable vehicles and solvents that can be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable media prior to use.

In order to prolong the effect of a drug, it is often desirable to slow the absorption of the drug from subcutaneous or intramuscular injection. This can be accomplished by the use of a liquid suspension or crystalline or amorphous material with poor water solubility. The rate of absorption of the drug then depends upon its rate of dissolution that, in turn, can depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include (poly(orthoesters) and poly(anhydrides). Depot injectable formulations are also prepared by entrapping the drug in liposomes or microemulsions which are compatible with body tissues.

Compositions for rectal or vaginal administration are preferably suppositories which can be prepared by mixing the compounds of this invention with suitable non-irritating excipients or carriers such as cocoa butter, polyethylene glycol, or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcelhdose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monosteamte, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form can also comprise buffering agents.

Solid compositions of a similar type can also be employed as fillers in soft and hard-filled gelatin capsules using such excipients as lactose or milk sugar as well as high molecular weight polyethylene glycols, and the like. The solid dosage forms of tablets, dragees, capsules, pills, and granules can be prepared with coatings and shells such as enteric coatings and other coatings well known in the pharmaceutical formulating art. They can optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type can also be employed as fillers in soft and hard-filled gelatin capsules using such excipients as lactose or milk sugar as well as high molecular weight polethylene glycols, and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound can be admixed with at least one inert diluent such as sucrose, lactose and starch. Such dosage forms can also comprise, as in normal practice, additional substances other than inert diluents, e.g., tableting lubricants and other tableting aids such as magnesium stearate and microcrystalline cellulose. In the case of capsules, tablets and pills, the dosage forms can also comprise buffering agents. They can optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

The present invention encompasses pharmaceutically acceptable topical formulations of inventive compounds. The term “pharmaceutically acceptable topical formulation”, as used herein, means any formulation which is pharmaceutically acceptable for intradermal administration of a compound of the invention by application of the formulation to the epidermis. In certain embodiments of the invention, the topical formulation comprises a excipient system. Pharmaceutically effective excipients include, but are not limited to, solvents (e.g., alcohols, poly alcohols, water), creams, lotions, ointments, oils, plasters, liposomes, powders, emulsions, microemulsions, and buffered solutions (e.g., hypotonic or buffered saline) or any other excipient known in the art for topically administering pharmaceuticals. A more complete listing of art-known carvers is provided by reference texts that are standard in the art, for example, Remington's Pharmaceutical Sciences, 16th Edition, 1980 and 17th Edition, 1985, both published by Mack Publishing Company, Easton, Pa., the disclosures of which are incorporated herein by reference in their entireties. In certain other embodiments, the topical formulations of the invention can comprise excipients. Any pharmaceutically acceptable excipient known in the art can be used to prepare the inventive pharmaceutically acceptable topical formulations. Examples of excipients that can be included in the topical formulations of the invention include, but are not limited to, preservatives, antioxidants, moisturizers, emollients, buffering agents, solubilizing agents, other penetration agents, skin protectants, surfactants, and propellants, and/or additional therapeutic agents used in combination to the inventive compound. Suitable preservatives include, but are not limited to, alcohols, quaternary amines, organic acids, parabens, and phenols. Suitable antioxidants include, but are not limited to, ascorbic acid and its esters, sodium bisulfite, butylated hydroxytoluene, butylated hydroxyarrisole, tocopherols, and chelating agents like EDTA and citric acid. Suitable moisturizers include, but are not limited to, glycerine, sorbitol, polyethylene glycols, urea, and propylene glycol. Suitable buffering agents for use with the invention include, but are not limited to, citric, hydrochloric, and lactic acid buffers. Suitable solubilizing agents include, but are not limited to, quaternary ammonium chlorides, cyclodextrins, benzyl benzoate, lecithin, and polysorbates. Suitable skin protectants that can be used in the topical formulations of the invention include, but are not limited to, vitamin E oil, allatoin, dimethicone, glycerin, petrolatum, and zinc oxide.

In certain embodiments, the pharmaceutically acceptable topical formulations of the invention comprise at least a compound of the invention and a penetration enhancing agent. The choice of topical formulation will depend or several factors, including the condition to be treated, the physicochemical characteristics of the inventive compound and other excipients present, their stability in the formulation, available manufacturing equipment, and costs constraints. As used herein the term “penetration enhancing agent” means an agent capable of transporting a pharmacologically active compound through the stratum coreum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997). In certain exemplary embodiments, penetration agents for use with the invention include, but are not limited to, triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate), and N-methyl pyrrolidone.

In certain embodiments, the compositions can be in the form of ointments, pastes, creams, lotions, gels, powders, solutions, sprays, inhalants or patches. In certain exemplary embodiments, formulations of the compositions according to the invention are creams, which can further contain saturated or unsaturated fatty acids such as stearic acid, palmitic acid, oleic acid, palmito-oleic acid, cetyl or oleyl alcohols, stearic acid being particularly preferred. Creams of the invention can also contain a non-ionic surfactant, for example, polyoxy-40-stearate. In certain embodiments, the active component is admixed under sterile conditions with a pharmaceutically acceptable excipient and any needed preservatives or buffers as can be required. Ophthalmic formulation, eardrops, and eye drops are also contemplated as being within the scope of this invention. Additionally, the present invention contemplates the use of transdermal patches, which have the added advantage of providing controlled delivery of a compound to the body. Such dosage forms are made by dissolving or dispensing the compound in the proper medium. As discussed above, penetration enhancing agents can also be used to increase the flux of the compound across the skin. The rate can be controlled by either providing a rate controlling membrane or by dispersing the compound in a polymer matrix (e.g., PLGA) or gel.

In certain embodiments, the compositions of the invention can be used for external application, for example, ointment, cream, protector, adhesive skin patch, pack, toner, milky lotion, lotion, bath agent, hair lotion, hair tonic, hair liquid, shampoo and rinse are included. Further, an ingredient commonly used for an external agent, for example, whitening agent, moisturizer, oily ingredient, UV absorber, surfactant, thickening agent, alcohols, powder component, coloring material, aqueous ingredient, water, and various skin nutrients can be optionally blended in the agent for enhancing the expression of a redox-associated factor used in the present method. In addition, an auxiliary agent commonly used for an external preparation, for example, a metal-chelate agent such as disodium edetate, trisodium edetate, sodium citrate, sodium polyphosphates, sodium metaphosphate, gluconic acid; caffeine, tannin, verapamil, tranexamic acid and their derivatives; a medical agent such as licorice extract, glabridin, hot water extract of Chinese quince fruit, various herbal medicines, tocopherol acetate, glycyrrhizinate, and their derivatives, or their salts; a whitening agent such as vitamin C, ascorbic acid magnesium phosphate, ascorbic acid glucoside, arbutin, kojic acid; a saccharide such as glucose, fructose, mannose, sucrose, and trehalose; vitamin A such as retinoic acid, retinol, retinol acetate, and retinol palmitate can be optionally blended in the agent for inhibiting the expression of MITF used in the present method.

It will also be appreciated that the compounds and pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed can achieve a desired effect for the same disorder (for example, an inventive compound can be administered concurrently with another skin pigmentation regulating agent), or they can achieve different effects (e.g., control of an adverse effects).

For example, other therapies or skin pigmentation regulating agents that can be used in combination with the inventive compounds of the present invention for regulation of skin pigmentation include, but are not limited to, quinoline-based alkaloids from the cinchona tree, particularly quinine, chloroquinine; artemisinin; and aminoalkylphenol.

In certain embodiments, the pharmaceutical compositions of the present invention further comprise one or more additional therapeutically active ingredients (e.g., chemotherapeutic and/or palliative). For purposes of the invention, the term “palliative” refer, to treatment that is focused on the relief of symptoms of a disease and/or side effects of a therapeutic regimen, but is not curative.

Additionally, the present invention provides pharmaceutically acceptable derivatives of the skin pigmentation regulation compounds of the invention, and methods of treating a subject using these compounds, pharmaceutical compositions thereof, or either of these in combination with one or more additional therapeutic agents.

It will also be appreciated that certain of the compounds of present invention can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or a prodrug or other adduct or derivative of a compound of this invention which upon administration to a patient in need is capable of providing, directly or indirectly, a compound as otherwise described herein, or a metabolite or residue thereof.

The invention also provides prodrug forms of the above compounds, wherein the prodrug is metabolized in vivo to produce an analog or derivative as set forth above. Indeed, some of the above described analogs or derivatives can be a prodrug for another analog or derivative. The term “prodrug” is well understood in the art and includes compounds that are converted to pharmaceutically active compounds in a biological system. For example, see Remington's Pharmaceutical Sciences, 1980, vol. 16, Mack Publishing Company, Easton, Pa., 61 and 424.

Another aspect of the invention relates to a kit for conveniently and effectively carrying out the methods in accordance with the present invention. In general, the pharmaceutical pack or kit comprises one or more containers filled with one or more of the ingredients of the pharmaceutical compositions of the invention. Optionally associated with such container(s) can be a notice in the form prescribed by a governmental agency regulating the manufacture, use or sale of pharmaceutical products, which notice reflects approval by the agency of manufacture, use or sale for human administration.

### Pharmaceutical Uses and Methods of Treatment

In general, methods of using the compounds of the present invention comprise administering to a subject in need thereof a therapeutically effective amount of a compound of the present invention.

The compounds and pharmaceutical compositions of the present invention can be administered by an appropriate route. Suitable routes of administration include, but are not limited to, orally, intraperitoneally, subcutaneously, intramuscularly, transdermally, rectally, sublingualis intravenously, buccally, or inhalationally. Preferably, compounds and pharmaceutical compositions of the invention are administered topically. The pharmaceutical compositions of the invention preferably contain a pharmaceutically acceptable excipient suitable for rendering the compound or mixture administrable orally, parenterally, intravenously, intradermally, intramuscularly or subcutaneously, rectally, via inhalation or via buccal administration, or transdermally. The active ingredients can be admixed or compounded with a conventional, pharmaceutically acceptable excipient. It will be understood by those skilled in the art that a mode of administration, vehicle, excipient or carrier conventionally employed and which is inert with respect to the active agent can be utilized for preparing and administering the pharmaceutical compositions of the present invention. Illustrative of such methods, vehicles, excipients, and carriers are those described, for example, in Remington's Pharmaceutical Sciences, 18th ed. (1990), the disclosure of which is incorporated herein by reference. The formulations of the present invention for use in a subject comprise the agent, together with one or more acceptable excipient thereof, and optionally other therapeutic agents. The excipient must be “acceptable” in the sense of being compatible with the other ingredients of the formulation and not deleterious to the recipient thereof. The formulations can conveniently be presented in unit dosage form and can be prepared by any of the methods well known in the art of pharmacy. All methods include the step of bringing into association the agent with the excipient which constitutes one or more accessory ingredients. In general, the formulations are prepared by uniformly and intimately bringing into association the agent with the excipient and then, if necessary, dividing the product into unit dosages thereof.

Forms suitable for oral administration include tablets, troches, capsules, elixirs, suspensions, syrups, wafers, chewing gum, or the like prepared by art recognized procedures. The amount of active compound in such therapeutically useful compositions or preparations is such that a suitable dosage will be obtained. A syrup formulation will generally consist of a suspension or solution of the compound or salt in a liquid carrier, for example, ethanol, glycerine or water, with a flavoring or coloring agent. Where the composition is in the form of a tablet, one or more pharmaceutical excipient routinely used for preparing solid formulations can be employed. Examples of such excipient include magnesium stearate, starch, lactose and sucrose. Where the composition is in the form of a capsule, the use of routine encapsulation is generally suitable, for example, using the aforementioned excipient in a hard gelatin capsule shell. Where the composition is in the form of a soft gelatin shell capsule, pharmaceutical excipient routinely used for preparing dispersions or suspensions can be considered, for example, aqueous gums, celluloses, silicates, or oils, and are incorporated in a soft gelatin capsule shell.

Formulations suitable for parenteral administration conveniently include sterile aqueous preparations of the agents that are preferably isotonic with the blood of the recipient. Suitable excipient solutions include phosphate buffered saline, saline, water, lactated Ringer's or dextrose (5% in water). Such formulations can be conveniently prepared by admixing the agent with water to produce a solution or suspension, which is filled into a sterile container and sealed against bacterial contamination. Preferably, sterile materials are used under aseptic manufacturing conditions to avoid the need for terminal sterilization. Such formulations can optionally contain one or more additional ingredients, which can include preservatives such as methyl hydroxybenzoate, chlorocresol, metacresol, phenol and benzalkonium chloride. Such materials are of special value when the formulations are presented in multidose containers.

Buffers can also be included to provide a suitable pH value for the formulation. Suitable buffer materials include sodium phosphate and acetate. Sodium chloride or glycerin can be used to render a formulation isotonic with the blood.

If desired, a formulation can be filled into containers under an inert atmosphere such as nitrogen and can be conveniently presented in unit dose or multi-dose form, for example, in a sealed ampoule.

Those skilled in the art will be aware that the amounts of the various components of the compositions of the invention to be administered in accordance with the method of the invention to a subject will depend upon those factors noted above.

A typical suppository formulation includes the compound or a pharmaceutically acceptable salt thereof which is active when administered in this way, with a binding and/or lubricating agent, for example, polymeric glycols, gelatins, cocoa-butter, or other low melting vegetable waxes or fats. Typical transdermal formulations include a conventional aqueous or nonaqueous vehicle, for example, a cream, ointment, lotion, or paste or are in the form of a medicated plastic, patch or membrane.

Typical compositions for inhalation are in the form of a solution, suspension, or emulsion that can be administered in the form of an aerosol using a conventional propellant such as dichlorodifluoromethane or trichlorofluoromethane.

The therapeutically effective amount of a compound or pharmaceutical composition of the invention depends, in each case, upon several factors, e.g., the health, age, gender, size, and condition of the subject to be treated, the intended mode of administration, and the capacity of the subject to incorporate the intended dosage form, among others. A therapeutically effective amount of an active agent is an amount sufficient to have the desired effect for the condition being treated.

The term “treatment” as used herein includes any treatment of a condition or disease in an animal, particularly a mammal, more particularly a human, and includes: (i) preventing the disease or condition from occurring in a subject which has not yet been diagnosed as having it; (ii) inhibiting the disease or condition, i.e. arresting its development; relieving the disease or condition, i.e. causing regression of the condition; or relieving the conditions caused by the disease, i.e. symptoms of the disease. For example, in a method of regulating skin pigmentation, the desired effect is partial or total inhibition, delay or prevention of the progression of a disease or disorder whose regulation of skin pigmentation is an issue; inhibition, delay or prevention of the recurrence of a disease or disorder whose regulation of skin pigmentation is an issue; or the prevention of the onset or development of a disease or disorder whose regulation of skin pigmentation is an issue in a mammal, for example a human.

The term “therapeutically effective amount” refers to that amount which is sufficient to effect treatment, as defined herein, when administered to a mammal in need of such treatment. The therapeutically effective amount will vary depending on the subject and disease state being treated, the severity of the affliction and the manner of administration, and can be determined routinely by one of ordinary skill in the art.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the compound of the invention, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay. The dosage can be determined by a physician and adjusted, as necessary, to suit observed effects of the treatment.

In certain embodiments, an effective dose of a composition comprising a compound of the invention as described herein can be administered to a patient once. In certain embodiments, an effective dose of a composition comprising a compound of the invention can be administered to a patient repeatedly. Patients can be administered a therapeutic amount of a composition comprising a compound of the invention, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more. A composition comprising a compound of the invention can be administered over a period of time, such as over a 5 minute, 10 minute, 15 minute, 20 minute, or 25 minute period. If warranted, the administration can be repeated, for example, on a regular basis, such as hourly for 3 hours, 6 hours, 12 hours or longer or such as biweekly (i.e., every two weeks) for one month, two months, three months, four months or longer. In some instances, after an initial treatment regimen, the treatments can be administered on a less frequent basis. For example, after administration biweekly for three months, administration can be repeated once per month, for six months or a year or longer.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the compound of the invention.

In some embodiments, the technology described herein relates to a pharmaceutical composition comprising a compound of the invention as described herein, and optionally a pharmaceutically acceptable carrier. Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, sunflower oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent, e.g. a compound of the invention as described herein.

Without being bound by a theory, in certain embodiments, the compound of the invention selectively inhibits liver stage parasites.

In accordance with the present invention, the skin pigmentation can be regulated.

### DEFINITIONS

For convenience, certain terms employed herein, in the specification, examples and appended claims are collected here. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

The term “biologically active”, as used herein, refers to a protein having structural, regulatory, or biochemical functions of a naturally occurring molecule, and displays the activity of the molecule in a cellular and/or in vivo assay.

The term “isomer” as used herein refers to a compound with the same molecular formula but different structural formulas. Isomers do not necessarily share similar properties, unless they also have the same functional groups. There are many different classes of isomers, like stereoisomers, enantiomers, geometrical isomers, etc. There are two main forms of isomerism: structural isomerism and stereoisomerism (spatial isomerism).

The designations “R” and “S” are used to denote the absolute configuration of a molecule about its chiral center. The designations can appear as a prefix or as a suffix; they can or cannot be separated from the isomer by a hyphen; they can or cannot be hyphenated; and they can or cannot be surrounded by parentheses.

The term “S isomer” as used herein refers to an enantiomer with the chiral center S according to a system by which its substituents are each assigned a priority, according to the Cahn-Ingold-Prelog priority rules (CIP), based on atomic number, where the priority of atomic number decreases in counterclockwise direction, it is S enantiomer (from the Latin Sinestra, meaning “left”). Without wishing to be limited to theory, if the center is oriented so that the lowest-priority of the four is pointed away from a viewer, the viewer will then see two possibilities: If the priority of the remaining three substituents decreases in clockwise direction, it is labeled R (from the Latin Rectus, meaning “right”), if it decreases in counterclockwise direction, it is S (from the Latin Sinestra, meaning “left”).

The term “treating”, as used herein, refers to altering the disease course of the subject being treated. Therapeutic effects of treatment include, without limitation, preventing occurrence or recurrence of disease, alleviation of symptom(s), diminishment of direct or indirect pathological consequences of the disease, decreasing the rate of disease progression, amelioration or palliation of the disease state, and remission or improved prognosis.

The term “pharmaceutically acceptable excipient”, as used herein, refers to carriers and vehicles that are compatible with the active ingredient (for example, a compound of the invention) of a pharmaceutical composition of the invention (and preferably capable of stabilizing it) and not deleterious to the subject to be treated. For example, solubilizing agents that form specific, more soluble complexes with the compounds of the invention can be utilized as pharmaceutical excipients for delivery of the compounds. Suitable carriers and vehicles are known to those of extraordinary skill in the art. The term “excipient” as used herein will encompass all such carriers, adjuvants, diluents, solvents, or other inactive additives. Suitable pharmaceutically acceptable excipients include, but are not limited to, water, salt solutions, alcohol, vegetable oils, polyethylene glycols, gelatin, lactose, amylose, magnesium stearate, talc, silicic acid, viscous paraffin, perfume oil, fatty acid monoglycerides and diglycerides, petroethral fatty acid esters, hydroxymethyl-cellulose, polyvinylpyrrolidone, etc. The pharmaceutical compositions of the invention can also be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like, which do not deleteriously react with the active compounds of the invention.

The terms “salts” and “pharmaceutically acceptable salts” refer to organic and inorganic salts of a compound, a stereoisomer of a compound, or a prodrug of a compound as disclosed herein. Thus, as used herein, the term “pharmaceutically acceptable salt,” is a salt formed from an acid and a basic group of a compound of the invention. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate salts.

The term “pharmaceutically acceptable salt” also refers to a salt prepared from a compound as disclosed herein having an acidic functional group, such as a carboxylic acid functional group, and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl, N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-(hydroxymethyl)methylamine, N,N,-di-lower alkyl-N-(hydroxy lower alkyl)-amines, such as N,N-dimethyl-N-(2-hydroxyethyl)amine, or tri-(2-hydroxyethyl)amine; N-methyl-D-glucamine; and amino acids such as arginine, lysine, and the like. Other pharmaceutically acceptable salts are described in the Handbook of Pharmaceutical Salts. Properties, Selection, and Use (P. Heinrich Stahl and C. Wermuth, Eds., Verlag Helvetica Chica Acta, Zurich, Switzerland (2002)). In other embodiments, the term “pharmaceutically acceptable salt,” is a salt formed from an acid and a basic group of a compound of the invention. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate salts.

The term “subject” as used herein refers to a vertebrate, preferably a mammal, more preferably a primate, still more preferably a human. Mammals include, without limitation, humans, primates, wild animals, feral animals, farm animals, sports animals, and pets. In some embodiment, a subject includes domestic and commercial farm animal, for example, but not limited to, cattle, pigs, horses and other commercial animals. In some embodiments, a subject is a male subject, however, subjects also include female subjects as well as subjects who are transgendered female to male subjects. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. A patient or subject includes any subset of the foregoing, e.g., all of the above. In certain embodiments of the aspects described herein, the subject is a mammal, e.g., a primate, e.g., a human. The terms, “patient” and “subject” are used interchangeably herein. In addition, the methods and compositions described herein can be used to treat domesticated animals and/or pets.

The term “prodrug” refers to a compound that formulated as a precursor compound that, following administration, activates or releases the active component of the compound in vivo via a chemical or physiological process (e.g., upon being brought to physiological pH or through enzyme activity). A discussion of the synthesis and use of prodrugs is provided by Higuchi and Stella, Prodrugs as Novel Delivery Systems, vol. 14 of the ACS Symposium Series, and Bioreversible Carriers in Drug Design, ed. Edward B. Roche, American Pharmaceutical Association and Pergamon Press, 1987. Accordingly, the term “prodrug” refers to compounds that can be converted via some chemical or physiological process (e.g., enzymatic processes and metabolic hydrolysis) to an inactive form that can be activated in vivo by some co-compound or a specific environmental condition, e.g., pH etc. A prodrug can be inactive when administered to a subject, i.e. an ester, but is converted in vivo to an active compound, for example, by hydrolysis to the free carboxylic acid or free hydroxyl. The prodrug compound often offers advantages of solubility, tissue compatibility or delayed release in an organism. The term “prodrug” is also meant to include any covalently bonded carriers, which release the active compound in vivo when such prodrug is administered to a subject.

The term “therapeutically effective amount” as used herein refers to an amount sufficient to effect a beneficial or desired clinical result upon treatment. Therapeutically effective amounts will vary, as recognized by those skilled in the art, depending on the specific disease treated, the route of administration, the excipient selected, and the possibility of combination therapy.

Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents. Furthermore, therapeutically effective amounts will vary, as recognized by those skilled in the art, depending on the specific disease treated, the route of administration, the excipient selected, and the possibility of combination therapy.

The term “skin,” as used herein, refers to the epidermis and/or dermis of an animal.

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level (e.g., in the absence of a compound of the invention).

The terms “increased”, “increase” or “enhance” or “activate” are all used herein to generally mean an increase by a statistically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level (e.g., in the absence of a compound of the invention).

The term “statistically significant” or “significantly” refers to statistical significance and generally means at least two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

The term “substantially” as used herein means a proportion of at least about 60%, or preferably at least about 70% or at least about 80%, or at least about 90%, at least about 95%, at least about 97% or at least about 99% or more, or any integer between 70% and 100%.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

Other than in the operating examples, or where otherwise indicated, all numbers expressing quantities of ingredients or reaction conditions used herein should be understood as modified in all instances by the term “about.” The term “about” when used in connection with percentages can mean±1%.

In this application and the claims, the use of the singular includes the plural unless specifically stated otherwise. In addition, use of “or” means “and/or” unless stated otherwise. Moreover, the use of the term “including,” as well as other forms, such as “includes” and “included,” is not limiting. Also, terms such as “element” or “component” encompass both elements and components comprising one unit and elements and components that comprise more than one unit unless specifically stated otherwise.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology, and molecular biology can be found in The Merck Manual of Diagnosis and Therapy, 18th Edition, published by Merck Research Laboratories, 2006 (ISBN 0-911910-18-2); Robert S. Porter et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006. Definitions of common terms in molecular biology are found in Benjamin Lewin, Genes IX, published by Jones & Bartlett Publishing, 2007 (ISBN-13: 9780763740634); Kendrew et al. (eds.), The Encyclopedia of Molecular Biology, published by Blackwell Science Ltd., 1994 (ISBN 0-632-02182-9); and Robert A. Meyers (ed.), Maniatis et al., Molecular Cloning: A Laboratory Manual, Cold Spring Harbor Laboratory Press, Cold Spring Harbor, N.Y., USA (1982); Sambrook et al., Molecular Cloning: A Laboratory Manual (2 ed.), Cold Spring Harbor Laboratory Press, Cold Spring Harbor, N.Y., USA (1989); Davis et al., Basic Methods in Molecular Biology, Elsevier Science Publishing, Inc., New York, USA (1986); or Methods in Enzymology: Guide to Molecular Cloning Techniques Vol. 152, S. L. Berger and A. R. Kimmerl Eds., Academic Press Inc., San Diego, USA (1987); Current Protocols in Molecular Biology (CPMB) (Fred M. Ausubel, et al. ed., John Wiley and Sons, Inc.), Current Protocols in Protein Science (CPPS) (John E. Coligan, et. al., ed., John Wiley and Sons, Inc.) and Current Protocols in Immunology (CPI) (John E. Coligan, et. al., ed. John Wiley and Sons, Inc.), which are all incorporated by reference herein in their entireties.

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

### Screening Assay

In yet another aspect, disclosed herein is a method for identifying a compound that can modulate the expression or activity of MITF. Generally the method comprises contacting a cell with a test compound and comparing the expression of a reporter to control or reference. A change in the expression, amount, or activity of the reporter relative to a control or reference indicates that the compound can modulate the expression or activity of MITF. A control can be a sample that is not contacted with a compound. A control can be a sample that is treated with a known modulator of MITF expression or activity. This can serve as a positive control. A positive control can be a sample that is treated with a known inhibitor of MITF.

In embodiments of the assay, the cell is capable of producing MITF and comprises a reporter gene operably linked to a promoter of a direct transcriptional target of MITF. The cell can produce MITF from an endogenous gene. Alternatively, or in addition, a cell can be a transformed cell comprising an exogenous MITF gene.

As used herein, the term “reporter gene” refers to a gene that expresses a molecule that produces a detectable response or signal. As used herein, the term “detectable” refers to a molecule or an element or functional group in a molecule that allows for the detection, imaging, and/or monitoring of the presence the molecule. A detectable response generally refers to a change in, or occurrence of, a signal that is detectable either by observation or instrumentally. Without limitations, the expressed molecule can be detected directly or the molecule can produce a detectable signal in the presence of a reagent. Further, any available method for determining the amount of the reporter in a culture can be employed. In some embodiments, detectable response is an optical signal, i.e., the reporter is an optical reporter. Suitable optical reporters include, but are not limited to, fluorescent reporters and chemiluminescent groups.

In some embodiments, the detectable response is fluorescence or a change in fluorescence, e.g., a change in fluorescence intensity, fluorescence excitation or emission wavelength distribution, fluorescence lifetime, and/or fluorescence polarization. In some embodiments, the reporter gene expresses an enzyme which can catalyze the production of a detectable signal from a reagent.

In some embodiments, the reporter gene expresses luciferase.

In some embodiments, the reporter gene expresses a fluorescent protein. Examples of fluorescent proteins suitable for use include, but are not limited to, green fluorescent protein, red fluorescent protein (e.g., DsRed), yellow fluorescent protein, cyan fluorescent protein, blue fluorescent protein, and variants thereof (see, e.g., U.S. Pat. Nos. 6,403,374, 6,800,733, and 7,157,566). Specific examples of GFP variants include, but are not limited to, enhanced GFP (EGFP), destabilized EGFP, the GFP variants described in Doan et al, Mol. Microbiol, 55:1767-1781 (2005), the GFP variant described in Crameri et al, Nat. Biotechnol., 14:315319 (1996), the cerulean fluorescent proteins described in Rizzo et al, Nat. Biotechnol, 22:445 (2004) and Tsien, Annu. Rev. Biochem., 67:509 (1998), and the yellow fluorescent protein described in Nagal et al, Nat. Biotechnol., 20:87-90 (2002). DsRed variants are described in, e.g., Shaner et al, Nat. Biotechnol., 22:1567-1572 (2004), and include mStrawberry, mCherry, morange, mBanana, mHoneydew, and mTangerine. Additional DsRed variants are described in, e.g., Wang et al, Proc. Natl. Acad. Sci. U.S.A., 101:16745-16749 (2004) and include mRaspberry and mPlum. Further examples of DsRed variants include mRFPmars described in Fischer et al, FEBS Lett., 577:227-232 (2004) and mRFPruby described in Fischer et al, FEBS Lett, 580:2495-2502 (2006).

Other non-limiting list of fluorescent proteins includes AceGFP, AcGFP1, AmCyan1, AQ143, AsRed2, Azami-Green (mAG), Cerulean, Cerulean, Citrine, cOFP, CopGFP, Cyan, CyPet, Dronpa, DsRed/DsRed2/DsRed-Express, DsRed-Monomer, EBFP, ECFP, EGFP, Emerald, eqFP611, EYFP, GFPs, HcRed1, HcRed-tandem, J-Red, Kaede, KFP, KikGR, mBanana, mCFP, mCherry, mCitrine, mEosEP, mHoneydew, MiCy, mKO, mOrange, mPlum, mRaspberry, mRFP1, mStrawberry, mTangerine, mYFP, mYFP, mYFP, PA-GFP, PA-mRFP, PhiYFP, PS-CFP-2, Renilla, tdEosFP, tdTomato, T-Sapphire, TurboGFP, UV-T-Sapphire, Venus, YPet, ZsYellow1, and derivatives and analogs thereof. In one embodiment, the fluorescent protein is Green Fluorescent Protein (GFP).

Specific devices or methods known in the art for the detection of fluorescence, e.g., from fluorophores or fluorescent proteins, include, but are not limited to, in vivo near-infrared fluorescence (see, e.g., Frangioni, Curr. Opin. Chem. Biol, 7:626-634 (2003)), the Maestro™ in vivo fluorescence imaging system (Cambridge Research & Instrumentation, Inc.; Woburn, Mass.), in vivo fluorescence imaging using a flying-spot scanner (see, e.g., Ramanujam et al, IEEE Transactions on Biomedical Engineering, 48:1034-1041 (2001), and the like. Other methods or devices for detecting an optical response include, without limitation, visual inspection, CCD cameras, video cameras, photographic film, laser-scanning devices, fluorometers, photodiodes, quantum counters, epifluorescence microscopes, scanning microscopes, flow cytometers, fluorescence microplate readers, or signal amplification using photomultiplier tubes.

The reporter gene can be operably linked to a promoter which is a direct transcriptional target of MITF. By a “promoter of a direct transcriptional target of MITF” is meant any gene promoter that is modulated directly by MITF. This can be due to direct interaction of MITF with the promoter. In some embodiments, the promoter of a direct transcriptional target of MITF is a promoter of the TRPM1 gene.

For the assay, cell can be optionally allowed to grow for a period time before contacting with the test compound. In some embodiments, a practitioner can obtain cells that are already planted in the appropriate vessel and allowed to grow for a period of time. In other embodiments, the practitioner plates the cell in the appropriate vessel and allow the cells to grow for a period time, e.g., at least one day, at least two days, at least three days, at least four days, at least five days, at least six days, at least seven days or more before contacting with the test compound.

After the test compound has been in contact with the cell for a sufficient period of time, amount of reporter (e.g., expression or activity) is measured and compared to a control or reference. For example, contact time can be from seconds to days or weeks. The practitioner can optimized the contact time for obtaining an optimal signal-to-noise ratio, time constraints, amount of test compound to be tested, number of cells, test volume, availability of reagents for the assay, and the like.

As used herein, the term “test compound” refers to compounds and/or compositions that are to be screened for their ability to stimulate and/or increase and/or promote motor neuron survival. The test compounds can include a wide variety of different compounds, including chemical compounds and mixtures of chemical compounds, e.g., small organic or inorganic molecules; saccharines; oligosaccharides; polysaccharides; biological macromolecules, e.g., peptides, proteins, and peptide analogs and derivatives; peptidomimetics; nucleic acids; nucleic acid analogs and derivatives; an extract made from biological materials such as bacteria, plants, fungi, or animal cells; animal tissues; naturally occurring or synthetic compositions; and any combinations thereof. In some embodiments, the test compound is a small molecule.

The number of possible test compounds runs into millions. Methods for developing small molecule, polymeric and genome based libraries are described, for example, in Ding, et al. J Am. Chem. Soc. 124: 1594-1596 (2002) and Lynn, et al., J. Am. Chem. Soc. 123: 8155-8156 (2001). Commercially available compound libraries can be obtained from, e.g., ArQule, Pharmacopia, graffinity, Panvera, Vitas-M Lab, Biomol International and Oxford. These libraries can be screened using the screening devices and methods described herein. Chemical compound libraries such as those from NIH Roadmap, Molecular Libraries Screening Centers Network (MLSCN) can also be used. A comprehensive list of compound libraries can be found at www.broad.harvard.edu/chembio/platform/screening/compound_libraries/index.htm. A chemical library or compound library is a collection of stored chemicals usually used ultimately in high-throughput screening or industrial manufacture. The chemical library can consist in simple terms of a series of stored chemicals. Each chemical has associated information stored in some kind of database with information such as the chemical structure, purity, quantity, and physiochemical characteristics of the compound.

Depending upon the particular embodiment being practiced, the test compounds can be provided free in solution, or may be attached to a carrier, or a solid support, e.g., beads. A number of suitable solid supports may be employed for immobilization of the test compounds. Examples of suitable solid supports include agarose, cellulose, dextran (commercially available as, i.e., Sephadex, Sepharose) carboxymethyl cellulose, polystyrene, polyethylene glycol (PEG), filter paper, nitrocellulose, ion exchange resins, plastic films, polyaminemethylvinylether maleic acid copolymer, glass beads, amino acid copolymer, ethylene-maleic acid copolymer, nylon, silk, etc. Additionally, for the methods described herein, test compounds can be screened individually, or in groups. Group screening is particularly useful where hit rates for effective test compounds are expected to be low such that one would not expect more than one positive result for a given group.

The test compound can be tested at any desired concentration. For example, the test compound can be tested at a final concentration of from 0.01 nm to about 10 mM. Further, the test can be tested at 2 or more (e.g., 2, 3, 4, 5, 6, 7, 8, 9, 10 or more) different concentrations. This can be helpful if the test compound is active only in a range of concentration. When the test compound is tested at 2 or more different concentrations, the concentration difference can range from 10-10,000 fold (e.g., 10-5000 fold, 10-1000 fold, 10-500 fold, or 10-250 fold).

In some embodiments, the test compound is assayed more than once and selected if it reproducibly modulates MITF expression or activity.

In some embodiments, the assay further comprises the step of determining of the compound has scored on any other screens. This can be accomplished by looking at the various chemical databases that describe activity of compounds in various assays. This can help in identifying compounds that are unique to the present assay. For example, one can utilize the ChemBank analytical tool at the Broad Institute. ChemBank is a database of results for small molecule screens, regardless of the precise nature of screen, as long as they were carried out at the Broad Institute. In some embodiments, a compound is selected that scores in less than 3 (e.g., 3, 2, 1, or 0) other screens.

In some embodiments, selected test compound exhibits dose-dependent modulation of MITF expression or activity. In some embodiments, selected test compound exhibits maximal modulation of MITF expression or activity in the assay. This can be helpful because some highly potent modulators (based on IC50) can yield only weak maximal suppression, whereas other less potent modulators (based on IC50) can produce significantly greater suppression, even at doses below their IC50.

A drug's distribution coefficient strongly affects how easily the drug can reach its intended target in the body, how strong an effect it will have once it reaches its target, and how long it will remain in the body in an active form. Log P is one criterion used in medicinal chemistry to assess the druglikeness of a given molecule, and used to calculate lipophilic efficiency, a function of potency and Log P that evaluate the quality of research compounds. Log P value can be determined experimentally using methods well known in the art. Alternatively, or in addition, Log P value can be predicted from the chemical structure of the compound. Methods for predicting Log P values are also well known in the art. See for example, Ghose and Crippen (J. Comp. Chem. 9, 80(1988)).

Generally, a compound with Log P value in the range 0.7-5.9 penetrate the skin, however, skin penetration can be diminished if the log P_{OW} value is higher than 5.5. Accordingly, in some embodiments, the selected test compound has a Log P less than or equal to 6 (e.g., less than or equal to 5, less than or equal to 4, less than or equal to 3, less than or equal to 2, or less than or equal to 1), In some embodiments, the selected compound has a Log P value in the range from 0.5-6 (e.g., 0.5-5.5, 0.5

In one embodiment, the selected compound has a Log P value in the range from about 0.7 to about 6.

Without limitations, cells can be plated or cultured at any density that provides a optimal signal-to-noise ratio. For example, cells can be plated or cultured at a density of 1,000 to 20,000 cells/well in a 384-well plate. In some embodiments, motor neurons are plated at density of 1,000; 2,000; 4,000; 8,000; 12,000; 16,000; or 20,000 cells/well in a 384-well plate. Based on foregoing, one of ordinary skill can adjust the plating density for other cell culturing vessels. For example one can calculate the dimensions of a well in the 384-well plate and the vessels to be used and scale the number of cells to be plated based on volume or surface area ratio between a well from the 384-well plate and the vessel to be used.

The assay can be performed any suitable container or apparatus available to one of skill in the art for cell culturing. For example, the assay can be performed in 24-, 96-, or 384-well plates. In one embodiment, the assay is performed in a 384-well plate.

Cells for the aspects disclosed herein can be obtained from any source available to one of skill in the art. Additionally, cells can be of any origin. Accordingly, in some embodiments, the cell is from a mammalian source. In some embodiments, the cell is a melanocyte or from a melanoma cell line. In one embodiment, the cell is from the human melanoma cell line Skme15.

In some embodiments, the cell is from a subject, e.g., a patient. In some embodiments, the subject, e.g., a patient who is in need of regulation of skin pigmentation.

In some embodiments, the screening method is a high-throughput screening. High-throughput screening (HTS) is a method for scientific experimentation that uses robotics, data processing and control software, liquid handling devices, and sensitive detectors. High-Throughput Screening or HTS allows a researcher to quickly conduct millions of biochemical, genetic or pharmacological tests. High-Throughput Screening are well known to one skilled in the art, for example, those described in U.S. Pat. Nos. 5,976,813; 6,472,144; 6,692,856; 6,824,982; and 7,091,048, and contents of each of which is herein incorporated by reference in its entirety.

HTS uses automation to run a screen of an assay against a library of candidate compounds. An assay is a test for specific activity: usually inhibition or stimulation of a biochemical or biological mechanism. Typical HTS screening libraries or “decks” can contain from 100,000 to more than 2,000,000 compounds.

The key labware or testing vessel of HTS is the microtiter plate: a small container, usually disposable and made of plastic, which features a grid of small, open divots called wells. Modern microplates for HTS generally have either 384, 1536, or 3456 wells. These are all multiples of 96, reflecting the original 96 well microplate with 8×12 9 mm spaced wells.

To prepare for an assay, the researcher fills each well of the plate with the appropriate reagents that he or she wishes to conduct the experiment with, such as a motor neuron cell population. After some incubation time has passed to allow the reagent to absorb, bind to, or otherwise react (or fail to react) with the compounds in the wells, measurements are taken across all the plate's wells, either manually or by a machine. Manual measurements are often necessary when the researcher is using microscopy to (for example) seek changes that a computer could not easily determine by itself. Otherwise, a specialized automated analysis machine can run a number of experiments on the wells such as colorimetric measurements, radioactivity counting, etc. In this case, the machine outputs the result of each experiment as a grid of numeric values, with each number mapping to the value obtained from a single well. A high-capacity analysis machine can measure dozens of plates in the space of a few minutes like this, generating thousands of experimental data points very quickly.

In another aspect, the invention provides a compound selected by the screening assay described herein. It is to be understood that analogs, derivatives, isomers, and pharmaceutically acceptable salts of the compounds selected by the screening assays described herein are also claimed herein.

In some embodiments, the invention can be described as in any numbered paragraph:
1. A method for regulating skin pigmentation comprising: administering to a subject in need thereof, a pharmaceutical composition comprising a therapeutic agent inhibiting the expression or activation of Microphthalmia Transcription Factor (MITF).
2. The method of paragraph 1, wherein the therapeutic agent is selected from the group consisting of:
1-[(1S,3R,5R,7S)-3-(3,4-dimethylphenyl)adamantan-1-yl]methanamine;
5-chloro-7-[(2-chloro-6-fluorophenyl)(pyridin-2-ylamino)methyl]quinolin-8-ol;
(1S,2R,3S,6S)-1-benzoyl-6-tert-butylspiro[2.5]octane-1,2-dicarbonitrile;
N-[(4-chlorophenyl)(2-hydroxy-1-naphthyl)methyl]-3-methoxybenzamide;
Ethyl-1-benzyl-5-[3-(tert-butylamino)-2-hydroxypropoxy]-2-methyl-1H-indole-3-carboxylate;
5-chloro-7-[(2,5-dimethylphenyl)(pyridin-2-ylamino)methyl]quinolin-8-ol;
N-[5-(2,3-dihydro-1,4-benzodioxin-6-yl)-2-methyl-3-furoyl]glycine;
[3-(1,3-benzothiazol-2-yl)phenoxy]acetic acid;
N-(2-fluorophenyl)-2-{[5-(3-methylphenyl)-1,3,4-oxadiazol-2-yl]thio}acetamide;
N-[4-(6-methyl-1,3-benzothiazol-2-yl)phenyl]-2-[(5-methyl-1,3,4-thiadiazol-2-yl)thio]acetamide;
2-(2-bromo-4-tert-butylphenoxy)-N-(2-methoxy-5-nitrophenyl)acetamide; Methyl-5-{[(4-fluoro-2-methylphenyl)sulfonyl](isonicotinoyl)amino}-2-methyl-1-benzofuran-3-carboxylate;
1-(4-chlorobenzyl)-N-(pyridin-3-ylmethyl)-1H-pyrazolo[3,4-d]pyrimidin-4-amine;
2-{[5-(4-chlorophenyl)-4-(3-methylphenyl)-4H-1,2,4-triazol-3-yl]thio}-N-(3-methoxyphenyl)acetamide;
N-phenyl-3-[(4-phenyl-1,3-thiazol-2-yl)thio]propanamide;
Ethyl-[3-(1,3-benzothiazol-2-yl)phenoxy]acetate;
2-methyl-3-[(4-methylpiperidin-1-yl)carbonyl]naphtho[2,3-b]furan-4,9-dione;
N-(3-phenylpropyl)-10H-phenothiazine-10-carboxamide;
5-(4-chlorophenyl)-2-pyrrolidin-1-yl-6H-1,3,4-thiadiazine;
7-(4-fluorophenyl)-1-(2-furylmethyl)-5,5-bis(trifluoromethyl)-5,8-dihydropyrimido[4,5-d]pyrimidine-2,4(1H,3H)-dione;
3-{[(2-bromo-4-methylphenoxy)acetyl]amino}benzoic acid;
3-{[4-(hydroxymethyl)-2-methoxyphenoxy]methyl}benzoic acid;
5-phenyl[1,2,5]oxadiazolo[3,4-b]pyrazine;
2,3,9-trimethoxy-6a,12a-dihydrochromoeo[3,4-b]chromen-12(6H)-one;
4-[(4-chlorobenzyl)oxy]-3-methoxy-N-(2-methyl-5-[1,3]oxazolo[4,5-b]pyridin-2-ylphenyl)benzamide;
3-(1,3-benzothiazol-2-ylthio)propanoic acid;
3-{[(4-ethoxyphenoxy)acetyl]amino}benzoic acid;
N-[4-chloro-3-(trifluoromethyl)phenyl]-1-[(2,5-dimethoxyphenyl)sulfonyl]piperidine-4-carboxamide;
N-[3,5-bis(trifluoromethyl)phenyl]-2-[(5-nitro-1H-benzimidazol-2-yl)thio]acetamide;
5-[4-(4-tert-butylbenzoyl)piperazin-1-yl]-2-nitro-N-(pyridin-3-ylmethyl)aniline;
3-{[2-chloro-4-(hydroxymethyl)phenoxy]methyl}benzoic acid;
{[(2-chlorophenyl)amino][(3-fluorobenzyl)thio]methylene}malononitrile;
{[(4-bromophenyl)amino][(4-fluorobenzyl)thio]methylene}malononitrile; and
1-[(1S,3R,5R,7S)-3-(3,4-dimethylphenyl)adamantan-1-yl]methanamine;
(3,5-di-t-butyl-4-hydroxybenzylidene)malononitrile;
2-amino-N-{5-[6-(dimethylamino)-9H-purin-9-yl]-4-hydroxy-2-(hydroxymethyl)tetrahydrofuran-3-yl}-3-(4-methoxyphenyl)propanamide;
1,3,6-trihydroxy-7-methoxy-2,8-bis(3-methylbut-2-en-1-yl)-9H-xanthen-9-one;
(3S,3aR,4R,6S,6aR,7S,8R,9bR)-6-acetoxy-4-(butyryloxy)-3,3a-dihydroxy-3,6,9-trimethyl-8-{[(2Z)-2-methylbut-2-enoyl]oxy}-2-oxo-2,3,3a,4,5,6,6a,7,8,9b-decahydroazuleno[4,5-b]furan-7-ylhexanoate;
(1E,5S)-6,6,6-trifluoro-5-hydroxy-1,5-diphenylhex-1-en-3-one;
2-(4-methoxyphenyl)-1,3-benzothiazole;
Ethyl-4-amino-2-{[(1E)-3-(4-methylphenyl)-3-oxoprop-1-en-1-yl]thio}pyrimidine-5-carboxylate;
3-[(5-chloro-1,3-benzothiazol-2-yl)thio]-5,5-dimethylcyclohex-2-en-1-one;
(2E)-3-[(5-methyl-1H-benzimidazol-2-yl)thio]-1-phenylprop-2-en-1-one;
11,12-dihydroxy-3-isobutyl-4,5,8-trimethyl-3,3a,4,6a,9,10,11,12-octahydro-1H-cycloundeca[d]isoindole-1,15 (2H)-dione;
3-(hydroxymethyl)-6-isopropyl-5-[(2-methylbut-2-enoyl)oxy]-2-oxocyclohex-3-en-1-yl4-hydroxy-2-methylbut-2-enoate;
1,11,12-trihydroxy-2,16-dioxo-11,20-epoxypicras-3-en-15-yl2-hydroxy-2-methylbutanoate;
1-[3,5-bis(trifluoromethyl)phenyl]-3-[3-chloro-4-(isopropylsulfonyl)-2-thienyl]urea;
   a pharmaceutical salt thereof;
   and any combination thereof.
   3. The method of paragraph 2, wherein the therapeutic agent is selected from the group consisting of:
1-[(1S,3R,5R,7S)-3-(3,4-dimethylphenyl)adamantan-1-yl]methanamine;
5-chloro-7-[(2-chloro-6-fluorophenyl)(pyridin-2-ylamino)methyl]quinolin-8-ol;
(1S,2R,3S,6S)-1-benzoyl-6-tert-butylspiro[2.5]octane-1,2-dicarbonitrile;
N-[(4-chlorophenyl)(2-hydroxy-1-naphthyl)methyl]-3-methoxybenzamide;
Ethyl-1-benzyl-5-[3-(tert-butylamino)-2-hydroxypropoxy]-2-methyl-1H-indole-3-carboxylate;
5-chloro-7-[(2,5-dimethylphenyl)(pyridin-2-ylamino)methyl]quinolin-8-ol;
N-[5-(2,3-dihydro-1,4-benzodioxin-6-yl)-2-methyl-3-furoyl]glycine;
[3-(1,3-benzothiazol-2-yl)phenoxy]acetic acid;
N-(2-fluorophenyl)-2-{[5-(3-methylphenyl)-1,3,4-oxadiazol-2-yl]thio}acetamide;
N-[4-(6-methyl-1,3-benzothiazol-2-yl)phenyl]-2-[(5-methyl-1,3,4-thiadiazol-2-yl)thio]acetamide;
2-(2-bromo-4-tert-butylphenoxy)-N-(2-methoxy-5-nitrophenyl)acetamide;
Methyl-5-{[(4-fluoro-2-methylphenyl)sulfonyl](isonicotinoyl)amino}-2-methyl-1-benzofuran-3-carboxylate;
1-(4-chlorobenzyl)-N-(pyridin-3-ylmethyl)-1H-pyrazolo[3,4-d]pyrimidin-4-amine;
2-{[5-(4-chlorophenyl)-4-(3-methylphenyl)-4H-1,2,4-triazol-3-yl]thio}-N-(3-methoxyphenyl)acetamide;
N-phenyl-3-[(4-phenyl-1,3-thiazol-2-yl)thio]propanamide;
Ethyl-[3-(1,3-benzothiazol-2-yl)phenoxy]acetate;
2-methyl-3-[(4-methylpiperidin-1-yl)carbonyl]naphtho[2,3-b]furan-4,9-dione;
N-(3-phenylpropyl)-10H-phenothiazine-10-carboxamide;
5-(4-chlorophenyl)-2-pyrrolidin-1-yl-6H-1,3,4-thiadiazine;
7-(4-fluorophenyl)-1-(2-furylmethyl)-5,5-bis(trifluoromethyl)-5,8-dihydropyrimido[4,5-d]pyrimidine-2,4(1H,3H)-dione;
3-{[(2-bromo-4-methylphenoxy)acetyl]amino}benzoic acid;
3-{[4-(hydroxymethyl)-2-methoxyphenoxy]methyl}benzoic acid;
5-phenyl[1,2,5]oxadiazolo[3,4-b]pyrazine;
2,3,9-trimethoxy-6a,12a-dihydrochromoeo[3,4-b]chromen-12(6H)-one;
4-[(4-chlorobenzyl)oxy]-3-methoxy-N-(2-methyl-5-[1,3]oxazolo[4,5-b]pyridin-2-ylphenyl)benzamide;
3-(1,3-benzothiazol-2-ylthio)propanoic acid;
3-{[(4-ethoxyphenoxy)acetyl]amino}benzoic acid;
N-[4-chloro-3-(trifluoromethyl)phenyl]-1-[(2,5-dimethoxyphenyl)sulfonyl]piperidine-4-carboxamide;
N-[3,5-bis(trifluoromethyl)phenyl]-2-[(5-nitro-1H-benzimidazol-2-yl)thio]acetamide;
5-[4-(4-tert-butylbenzoyl)piperazin-1-yl]-2-nitro-N-(pyridin-3-ylmethyl)aniline;
3-{[2-chloro-4-(hydroxymethyl)phenoxy]methyl}benzoic acid;
{[(2-chlorophenyl)amino][(3-fluorobenzyl)thio]methylene}malononitrile;
{[(4-bromophenyl)amino][(4-fluorobenzyl)thio]methylene}malononitrile; and
1-[(1S,3R,5R,7S)-3-(3,4-dimethylphenyl)adamantan-1-yl]methanamine;
   a pharmaceutically acceptable salt thereof;
   and any combination thereof.
   4. The method of paragraph 2, wherein the therapeutic agent is selected from the group consisting of:
(3,5-di-t-butyl-4-hydroxybenzylidene)malononitrile;
2-amino-N-{5-[6-(dimethylamino)-9H-purin-9-yl]-4-hydroxy-2-(hydroxymethyl)tetrahydrofuran-3-yl}-3-(4-methoxyphenyl)propanamide;
1,3,6-trihydroxy-7-methoxy-2,8-bis(3-methylbut-2-en-1-yl)-9H-xanthen-9-one;
(3S,3aR,4R,6S,6aR,7S,8R,9bR)-6-acetoxy-4-(butyryloxy)-3,3a-dihydroxy-3,6,9-trimethyl-8-{[(2Z)-2-methylbut-2-enoyl]oxy}-2-oxo-2,3,3a,4,5,6,6a,7,8,9b-decahydroazuleno[4,5-b]furan-7-ylhexanoate;
(1E,5S)-6,6,6-trifluoro-5-hydroxy-1,5-diphenylhex-1-en-3-one;
2-(4-methoxyphenyl)-1,3-benzothiazole;
Ethyl-4-amino-2-{[(1E)-3-(4-methylphenyl)-3-oxoprop-1-en-1-yl]thio}pyrimidine-5-carboxylate;
3-[(5-chloro-1,3-benzothiazol-2-yl)thio]-5,5-dimethylcyclohex-2-en-1-one;
(2E)-3-[(5-methyl-1H-benzimidazol-2-yl)thio]-1-phenylprop-2-en-1-one;
11,12-dihydroxy-3-isobutyl-4,5,8-trimethyl-3,3a,4,6a,9,10,11,12-octahydro-1H-cycloundeca[d]isoindole-1,15(2H)-dione;
3-(hydroxymethyl)-6-isopropyl-5-[(2-methylbut-2-enoyl)oxy]-2-oxocyclohex-3-en-1-yl4-hydroxy-2-methylbut-2-enoate;
1,11,12-trihydroxy-2,16-dioxo-11,20-epoxypicras-3-en-15-yl2-hydroxy-2-methylbutanoate;
1-[3,5-bis(trifluoromethyl)phenyl]-3-[3-chloro-4-(isopropylsulfonyl)-2-thienyl]urea;
   a pharmaceutically acceptable salt thereof;
   and any combination thereof.
   5. The method of paragraph 2, wherein the therapeutic agent is selected from the group consisting of:
1,3,6-trihydroxy-7-methoxy-2,8-bis(3-methylbut-2-en-1-yl)-9H-xanthen-9-one;
(1E,5S)-6,6,6-trifluoro-5-hydroxy-1,5-diphenylhex-1-en-3-one;
3-(hydroxymethyl)-6-isopropyl-5-[(2-methylbut-2-enoyl)oxy]-2-oxocyclohex-3-en-1-yl-4-hydroxy-2-methylbut-2-enoate;
   a pharmaceutically acceptable salt thereof;
   and any combination thereof.
   6. The method of any one of paragraphs 1-4, wherein pharmaceutical composition comprises an additional therapeutic agent.
   7. The method of paragraph 1, wherein the pharmaceutical composition comprises 0.001%-99% of the therapeutic agent, inhibiting the expression or activation of MITF.
   8. The method of paragraph 1, wherein the pharmaceutical composition is formulated as a topical formulation.
   9. The method of paragraph 1, wherein the pharmaceutical composition is formulated as a parenteral formulation.
   10. The method of paragraph 1, wherein the pharmaceutical composition is formulated as an oral formulation.
   11. The method of paragraph 1, wherein the pharmaceutical composition is administered once daily.
   12. A pharmaceutical composition comprising:
1-[(1S,3R,5R,7S)-3-(3,4-dimethylphenyl)adamantan-1-yl]methanamine;
5-chloro-7-[(2-chloro-6-fluorophenyl)(pyridin-2-ylamino)methyl]quinolin-8-ol;
(1S,2R,3S,6S)-1-benzoyl-6-tert-butylspiro[2.5]octane-1,2-dicarbonitrile;
N-[(4-chlorophenyl)(2-hydroxy-1-naphthyl)methyl]-3-methoxybenzamide;
Ethyl-1-benzyl-5-[3-(tert-butylamino)-2-hydroxypropoxy]-2-methyl-1H-indole-3-carboxylate;
5-chloro-7-[(2,5-dimethylphenyl)(pyridin-2-ylamino)methyl]quinolin-8-ol;
N-[5-(2,3-dihydro-1,4-benzodioxin-6-yl)-2-methyl-3-furoyl]glycine;
[3-(1,3-benzothiazol-2-yl)phenoxy]acetic acid;
N-(2-fluorophenyl)-2-{[5-(3-methylphenyl)-1,3,4-oxadiazol-2-yl]thio}acetamide;
N-[4-(6-methyl-1,3-benzothiazol-2-yl)phenyl]-2-[(5-methyl-1,3,4-thiadiazol-2-yl)thio]acetamide;
2-(2-bromo-4-tert-butylphenoxy)-N-(2-methoxy-5-nitrophenyl)acetamide;
Methyl-5-{[(4-fluoro-2-methylphenyl)sulfonyl](isonicotinoyl)amino}-2-methyl-1-benzofuran-3-carboxylate;
1-(4-chlorobenzyl)-N-(pyridin-3-ylmethyl)-1H-pyrazolo[3,4-d]pyrimidin-4-amine;
2-{[5-(4-chlorophenyl)-4-(3-methylphenyl)-4H-1,2,4-triazol-3-yl]thio}-N-(3-methoxyphenyl)acetamide;
N-phenyl-3-[(4-phenyl-1,3-thiazol-2-yl)thio]propanamide;
Ethyl-[3-(1,3-benzothiazol-2-yl)phenoxy]acetate;
2-methyl-3-[(4-methylpiperidin-1-yl)carbonyl]naphtho[2,3-b]furan-4,9-dione;
N-(3-phenylpropyl)-10H-phenothiazine-10-carboxamide;
5-(4-chlorophenyl)-2-pyrrolidin-1-yl-6H-1,3,4-thiadiazine;
7-(4-fluorophenyl)-1-(2-furylmethyl)-5,5-bis(trifluoromethyl)-5,8-dihydropyrimido[4,5-d]pyrimidine-2,4(1H,3H)-dione;
3-{[(2-bromo-4-methylphenoxy)acetyl]amino}benzoic acid;
3-{[4-(hydroxymethyl)-2-methoxyphenoxy]methyl}benzoic acid;
5-phenyl[1,2,5]oxadiazolo[3,4-b]pyrazine;
2,3,9-trimethoxy-6a,12a-dihydrochromoeo[3,4-b]chromen-12(6H)-one;
4-[(4-chlorobenzyl)oxy]-3-methoxy-N-(2-methyl-5-[1,3]oxazolo[4,5-b]pyridin-2-ylphenyl)benzamide;
3-(1,3-benzothiazol-2-ylthio)propanoic acid;
3-{[(4-ethoxyphenoxy)acetyl]amino}benzoic acid;
N-[4-chloro-3-(trifluoromethyl)phenyl]-1-[(2,5-dimethoxyphenyl)sulfonyl]piperidine-4-carboxamide;
N-[3,5-bis(trifluoromethyl)phenyl]-2-[(5-nitro-1H-benzimidazol-2-yl)thio]acetamide;
5-[4-(4-tert-butylbenzoyl)piperazin-1-yl]-2-nitro-N-(pyridin-3-ylmethyl)aniline;
3-{[(2-chloro-4-(hydroxymethyl)phenoxy]methyl}benzoic acid;
{[(2-chlorophenyl)amino][(3-fluorobenzyl)thio]methylene}malononitrile;
{[(4-bromophenyl)amino][(4-fluorobenzyl)thio]methylene}malononitrile; and
1-[(1S,3R,5R,7S)-3-(3,4-dimethylphenyl)adamantan-1-yl]methanamine;
(3,5-di-t-butyl-4-hydroxybenzylidene)malononitrile;
2-amino-N-{5-[6-(dimethylamino)-9H-purin-9-yl]-4-hydroxy-2-(hydroxymethyl)tetrahydrofuran-3-yl}-3-(4-methoxyphenyl)propanamide;
1,3,6-trihydroxy-7-methoxy-2,8-bis(3-methylbut-2-en-1-yl)-9H-xanthen-9-one;
(3S,3aR,4R,6S,6aR,7S,8R,9bR)-6-acetoxy-4-(butyryloxy)-3,3a-dihydroxy-3,6,9-trimethyl-8-{[(2Z)-2-methylbut-2-enoyl]oxy}-2-oxo-2,3,3a,4,5,6,6a,7,8,9b-decahydroazuleno[4,5-b]furan-7-ylhexanoate;
(1E,5S)-6,6,6-trifluoro-5-hydroxy-1,5-diphenylhex-1-en-3-one;
2-(4-methoxyphenyl)-1,3-benzothiazole;
Ethyl-4-amino-2-{[(1E)-3-(4-methylphenyl)-3-oxoprop-1-en-1-yl]thio}pyrimidine-5-carboxylate;
3-[(5-chloro-1,3-benzothiazol-2-yl)thio]-5,5-dimethylcyclohex-2-en-1-one;
(2E)-3-[(5-methyl-1H-benzimidazol-2-yl)thio]-1-phenylprop-2-en-1-one;
11,12-dihydroxy-3-isobutyl-4,5,8-trimethyl-3,3a,4,6a,9,10,11,12-octahydro-1H-cycloundeca[d]isoindole-1,15(2H)-dione;
3-(hydroxymethyl)-6-isopropyl-5-[(2-methylbut-2-enoyl)oxy]-2-oxocyclohex-3-en-1-yl4-hydroxy-2-methylbut-2-enoate;
1,11,12-trihydroxy-2,16-dioxo-11,20-epoxypicras-3-en-15-yl2-hydroxy-2-methylbutanoate;
1-[3,5-bis(trifluoromethyl)phenyl]-3-[3-chloro-4-(isopropylsulfonyl)-2-thienyl]urea;
   a pharmaceutically acceptable salt thereof;
   and any combination thereof.
   13. The pharmaceutical composition of paragraph 12, comprising:
1-[(1S,3R,5R,7S)-3-(3,4-dimethylphenyl) adamantan-1-yl]methanamine;
5-chloro-7-[(2-chloro-6-fluorophenyl)(pyridin-2-ylamino)methyl]quinolin-8-ol;
(1S,2R,3 S,6S)-1-benzoyl-6-tert-butylspiro[2.5]octane-1,2-dicarbonitrile;
N-[(4-chlorophenyl) (2-hydroxy-1-naphthyl)methyl]-3-methoxybenzamide;
Ethyl-1-benzyl-5-[3-(tert-butylamino)-2-hydroxypropoxy]-2-methyl-1H-indole-3-carboxylate;
5-chloro-7-[(2,5-dimethylphenyl)(pyridin-2-ylamino)methyl]quinolin-8-ol;
N-[5-(2,3-dihydro-1,4-benzodioxin-6-yl)-2-methyl-3-furoyl]glycine;
[3-(1,3-benzothiazol-2-yl)phenoxy]acetic acid;
N-(2-fluorophenyl)-2-{[5-(3-methylphenyl)-1,3,4-oxadiazol-2-yl]thio}acetamide;
N-[4-(6-methyl-1,3-benzothiazol-2-yl)phenyl]-2-[(5-methyl-1,3,4-thiadiazol-2-yl)thio]acetamide;
2-(2-bromo-4-tert-butylphenoxy)-N-(2-methoxy-5-nitrophenyl)acetamide;
Methyl-5-{[(4-fluoro-2-methylphenyl)sulfonyl](isonicotinoyl)amino}-2-methyl-1-benzofuran-3-carboxylate;
1-(4-chlorobenzyl)-N-(pyridin-3-ylmethyl)-1H-pyrazolo[3,4-d]pyrimidin-4-amine;
2-{[5-(4-chlorophenyl)-4-(3-methylphenyl)-4H-1,2,4-triazol-3-yl]thio}-N-(3-methoxyphenyl)acetamide;
N-phenyl-3-[(4-phenyl-1,3-thiazol-2-yl)thio]propanamide;
Ethyl-[3-(1,3-benzothiazol-2-yl)phenoxy]acetate;
2-methyl-3-[(4-methylpiperidin-1-yl)carbonyl]naphtho[2,3-b]furan-4,9-dione;
N-(3-phenylpropyl)-10H-phenothiazine-10-carboxamide;
5-(4-chlorophenyl)-2-pyrrolidin-1-yl-6H-1,3,4-thiadiazine;
7-(4-fluorophenyl)-1-(2-furylmethyl)-5,5-bis(trifluoromethyl)-5,8-dihydropyrimido[4,5-d]pyrimidine-2,4(1H,3H)-dione;
3-{[(2-bromo-4-methylphenoxy)acetyl]amino}benzoic acid;
3-{[4-(hydroxymethyl)-2-methoxyphenoxy]methyl}benzoic acid;
5-phenyl[1,2,5]oxadiazolo[3,4-b]pyrazine;
2,3,9-trimethoxy-6a,12a-dihydrochromoeo[3,4-b]chromen-12(6H)-one;
4-[(4-chlorobenzyl)oxy]-3-methoxy-N-(2-methyl-5-[1,3]oxazolo[4,5-b]pyridin-2-ylphenyl)benzamide;
3-(1,3-benzothiazol-2-ylthio)propanoic acid;
3-{[(4-ethoxyphenoxy)acetyl]amino}benzoic acid;
N-[4-chloro-3-(trifluoromethyl)phenyl]-1-[(2,5-dimethoxyphenyl)sulfonyl]piperidine-4-carboxamide;
N-[3,5-bis(trifluoromethyl)phenyl]-2-[(5-nitro-1H-benzimidazol-2-yl)thio]acetamide;
5-[4-(4-tert-butylbenzoyl)piperazin-1-yl]-2-nitro-N-(pyridin-3-ylmethyl) aniline;
3-{[2-chloro-4-(hydroxymethyl)phenoxy]methyl}benzoic acid;
{[(2-chlorophenyl)amino][(3-fluorobenzyl)thio]methylene}malononitrile;
{[(4-bromophenyl)amino][(4-fluorobenzyl)thio]methylene}malononitrile; and
1-[(1S,3R,5R,7 S)-3-(3,4-dimethylphenyl)adamantan-1-yl]methanamine;
   a pharmaceutically acceptable salt thereof;
   and any combination thereof.
   14. The pharmaceutical composition of paragraph 12, comprising:
(3,5-di-t-butyl-4-hydroxybenzylidene)malononitrile;
2-amino-N-{5-[6-(dimethylamino)-9H-purin-9-yl]-4-hydroxy-2-(hydroxymethyl)tetrahydrofuran-3-yl}-3-(4-methoxyphenyl)propanamide;
1,3,6-trihydroxy-7-methoxy-2,8-bis(3-methylbut-2-en-1-yl)-9H-xanthen-9-one;
(3S,3aR,4R,6S,6aR,7S,8R,9bR)-6-acetoxy-4-(butyryloxy)-3,3a-dihydroxy-3,6,9-trimethyl-8-{[(2Z)-2-methylbut-2-enoyl]oxy}-2-oxo-2,3,3a,4,5,6,6a,7,8,9b-decahydroazuleno[4,5-b]furan-7-ylhexanoate;
(1E,5S)-6,6,6-trifluoro-5-hydroxy-1,5-diphenylhex-1-en-3-one;
2-(4-methoxyphenyl)-1,3-benzothiazole;
Ethyl-4-amino-2-{[(1E)-3-(4-methylphenyl)-3-oxoprop-1-en-1-yl]thio}pyrimidine-5-carboxylate;
3-[(5-chloro-1,3-benzothiazol-2-yl)thio]-5,5-dimethylcyclohex-2-en-1-one;
(2E)-3-[(5-methyl-1H-benzimidazol-2-yl)thio]-1-phenylprop-2-en-1-one;
11,12-dihydroxy-3-isobutyl-4,5,8-trimethyl-3,3a,4,6a,9,10,11,12-octahydro-1H-cycloundeca[d]isoindole-1,15(2H)-dione;
3-(hydroxymethyl)-6-isopropyl-5-[(2-methylbut-2-enoyl)oxy]-2-oxocyclohex-3-en-1-yl4-hydroxy-2-methylbut-2-enoate;
1,11,12-trihydroxy-2,16-dioxo-11,20-epoxypicras-3-en-15-yl2-hydroxy-2-methylbutanoate;
1-[3,5-bis(trifluoromethyl)phenyl]-3-[3-chloro-4-(isopropylsulfonyl)-2-thienyl]urea;
   a pharmaceutically acceptable salt thereof;
   and any combination thereof.
   15. The pharmaceutical composition of paragraph 12, comprising:
1,3,6-trihydroxy-7-methoxy-2,8-bis(3-methylbut-2-en-1-yl)-9H-xanthen-9-one;
(1E,5S)-6,6,6-trifluoro-5-hydroxy-1,5-diphenylhex-1-en-3-one;
3-(hydroxymethyl)-6-isopropyl-5-[(2-methylbut-2-enoyl)oxy]-2-oxocyclohex-3-en-1-yl-4-hydroxy-2-methylbut-2-enoate;
   a pharmaceutically acceptable salt thereof;
   and any combination thereof.
   16. The pharmaceutical composition of any one of paragraphs 12-15, wherein the composition further comprises an additional therapeutic agent.
   17. A compound selected from:
1-[(1S,3R,5R,7S)-3-(3,4-dimethylphenyl)adamantan-1-yl]methanamine;
5-chloro-7-[(2-chloro-6-fluorophenyl)(pyridin-2-ylamino)methyl]quinolin-8-ol;
(1S,2R,3S,6S)-1-benzoyl-6-tert-butylspiro[2.5]octane-1,2-dicarbonitrile;
N-[(4-chlorophenyl)(2-hydroxy-1-naphthyl)methyl]-3-methoxybenzamide;
Ethyl-1-benzyl-5-[3-(tert-butylamino)-2-hydroxypropoxy]-2-methyl-1H-indole-3-carboxylate;
5-chloro-7-[(2,5-dimethylphenyl)(pyridin-2-ylamino)methyl]quinolin-8-ol;
N-[5-(2,3-dihydro-1,4-benzodioxin-6-yl)-2-methyl-3-furoyl]glycine;
[3-(1,3-benzothiazol-2-yl)phenoxy]acetic acid;
N-(2-fluorophenyl)-2-{[5-(3-methylphenyl)-1,3,4-oxadiazol-2-yl]thio}acetamide;
N-[4-(6-methyl-1,3-benzothiazol-2-yl)phenyl]-2-[(5-methyl-1,3,4-thiadiazol-2-yl)thio]acetamide;
2-(2-bromo-4-tert-butylphenoxy)-N-(2-methoxy-5-nitrophenyl)acetamide;
Methyl-5-{[(4-fluoro-2-methylphenyl)sulfonyl](isonicotinoyl)amino}-2-methyl-1-benzofuran-3-carboxylate;
1-(4-chlorobenzyl)-N-(pyridin-3-ylmethyl)-1H-pyrazolo[3,4-d]pyrimidin-4-amine;
2-{[5-(4-chlorophenyl)-4-(3-methylphenyl)-4H-1,2,4-triazol-3-yl]thio}-N-(3-methoxyphenyl)acetamide;
N-phenyl-3-[(4-phenyl-1,3-thiazol-2-yl)thio]propanamide;
Ethyl-[3-(1,3-benzothiazol-2-yl)phenoxy]acetate;
2-methyl-3-[(4-methylpiperidin-1-yl)carbonyl]naphtho[2,3-b]furan-4,9-dione;
N-(3-phenylpropyl)-10H-phenothiazine-10-carboxamide;
5-(4-chlorophenyl)-2-pyrrolidin-1-yl-6H-1,3,4-thiadiazine;
7-(4-fluorophenyl)-1-(2-furylmethyl)-5,5-bis(trifluoromethyl)-5,8-dihydropyrimido[4,5-d]pyrimidine-2,4(1H,3H)-dione;
3-{[(2-bromo-4-methylphenoxy)acetyl]amino}benzoic acid;
3-{[4-(hydroxymethyl)-2-methoxyphenoxy]methyl}benzoic acid;
5-phenyl[1,2,5]oxadiazolo[3,4-b]pyrazine;
2,3,9-trimethoxy-6a,12a-dihydrochromoeo[3,4-b]chromen-12(6H)-one;
4-[(4-chlorobenzyl)oxy]-3-methoxy-N-(2-methyl-5-[1,3]oxazolo[4,5-b]pyridin-2-ylphenyl)benzamide;
3-(1,3-benzothiazol-2-ylthio)propanoic acid;
3-{[(4-ethoxyphenoxy)acetyl]amino}benzoic acid;
N-[4-chloro-3-(trifluoromethyl)phenyl]-1-[(2,5-dimethoxyphenyl)sulfonyl]piperidine-4-carboxamide;
N-[3,5-bis(trifluoromethyl)phenyl]-2-[(5-nitro-1H-benzimidazol-2-yl)thio]acetamide;
5-[4-(4-tert-butylbenzoyl)piperazin-1-yl]-2-nitro-N-(pyridin-3-ylmethyl)aniline;
3-{[2-chloro-4-(hydroxymethyl)phenoxy]methyl}benzoic acid;
{[(2-chlorophenyl)amino][(3-fluorobenzyl)thio]methylene}malononitrile;
{[(4-bromophenyl)amino][(4-fluorobenzyl)thio]methylene}malononitrile; and
1-[(1S,3R,5R,7S)-3-(3,4-dimethylphenyl)adamantan-1-yl]methanamine;
(3,5-di-t-butyl-4-hydroxybenzylidene)malononitrile;
2-amino-N-{5-[6-(dimethylamino)-9H-purin-9-yl]-4-hydroxy-2-(hydroxymethyl)tetrahydrofuran-3-yl}-3-(4-methoxyphenyl)propanamide;
1,3,6-trihydroxy-7-methoxy-2,8-bis(3-methylbut-2-en-1-yl)-9H-xanthen-9-one;
(3S,3aR,4R,6S,6aR,7S,8R,9bR)-6-acetoxy-4-(butyryloxy)-3,3a-dihydroxy-3,6,9-trimethyl-8-{[(2Z)-2-methylbut-2-enoyl]oxy}-2-oxo-2,3,3a,4,5,6,6a,7,8,9b-decahydroazuleno[4,5-b]furan-7-ylhexanoate;
(1E,5S)-6,6,6-trifluoro-5-hydroxy-1,5-diphenylhex-1-en-3-one;
2-(4-methoxyphenyl)-1,3-benzothiazole;
Ethyl-4-amino-2-{[(1E)-3-(4-methylphenyl)-3-oxoprop-1-en-1-yl]thio}pyrimidine-5-carboxylate;
3-[(5-chloro-1,3-benzothiazol-2-yl)thio]-5,5-dimethylcyclohex-2-en-1-one;
(2E)-3-[(5-methyl-1H-benzimidazol-2-yl)thio]-1-phenylprop-2-en-1-one;
11,12-dihydroxy-3-isobutyl-4,5,8-trimethyl-3,3a,4,6a,9,10,11,12-octahydro-1H-cycloundeca[d]isoindole-1,15(2H)-dione;
3-(hydroxymethyl)-6-isopropyl-5-[(2-methylbut-2-enoyl)oxy]-2-oxocyclohex-3-en-1-yl4-hydroxy-2-methylbut-2-enoate;
1,11,12-trihydroxy-2,16-dioxo-11,20-epoxypicras-3-en-15-yl2-hydroxy-2-methylbutanoate;
1-[3,5-bis(trifluoromethyl)phenyl]-3-[3-chloro-4-(isopropylsulfonyl)-2-thienyl]urea;
   a pharmaceutically acceptable salt thereof;
   and any combination thereof for use in regulating skin pigmentation.
   18. The compound of paragraph 17 selected from:
1-[(1S,3R,5R,7S)-3-(3,4-dimethylphenyl)adamantan-1-yl]methanamine;
5-chloro-7-[(2-chloro-6-fluorophenyl)(pyridin-2-ylamino)methyl]quinolin-8-ol;
(1S,2R,3S,6S)-1-benzoyl-6-tert-butylspiro[2.5]octane-1,2-dicarbonitrile;
N-[(4-chlorophenyl)(2-hydroxy-1-naphthyl)methyl]-3-methoxybenzamide;
Ethyl-1-benzyl-5-[3-(tert-butylamino)-2-hydroxypropoxy]-2-methyl-1H-indole-3-carboxylate;
5-chloro-7-[(2,5-dimethylphenyl)(pyridin-2-ylamino)methyl]quinolin-8-ol;
N-[5-(2,3-dihydro-1,4-benzodioxin-6-yl)-2-methyl-3-furoyl]glycine;
[3-(1,3-benzothiazol-2-yl)phenoxy]acetic acid;
N-(2-fluorophenyl)-2-{[5-(3-methylphenyl)-1,3,4-oxadiazol-2-yl]thio}acetamide;
N-[4-(6-methyl-1,3-benzothiazol-2-yl)phenyl]-2-[(5-methyl-1,3,4-thiadiazol-2-yl)thio]acetamide;
2-(2-bromo-4-tert-butylphenoxy)-N-(2-methoxy-5-nitrophenyl)acetamide;
Methyl-5-{[(4-fluoro-2-methylphenyl)sulfonyl](isonicotinoyl)amino}-2-methyl-1-benzofuran-3-carboxylate;
1-(4-chlorobenzyl)-N-(pyridin-3-ylmethyl)-1H-pyrazolo[3,4-d]pyrimidin-4-amine;
2-{[5-(4-chlorophenyl)-4-(3-methylphenyl)-4H-1,2,4-triazol-3-yl]thio}-N-(3-methoxyphenyl)acetamide;
N-phenyl-3-[(4-phenyl-1,3-thiazol-2-yl)thio]propanamide;
Ethyl-[3-(1,3-benzothiazol-2-yl)phenoxy]acetate;
2-methyl-3-[(4-methylpiperidin-1-yl)carbonyl]naphtho[2,3-b]furan-4,9-dione;
N-(3-phenylpropyl)-10H-phenothiazine-10-carboxamide;
5-(4-chlorophenyl)-2-pyrrolidin-1-yl-6H-1,3,4-thiadiazine;
7-(4-fluorophenyl)-1-(2-furylmethyl)-5,5-bis(trifluoromethyl)-5,8-dihydropyrimido[4,5-d]pyrimidine-2,4(1H,3H)-dione;
3-{[(2-bromo-4-methylphenoxy)acetyl]amino}benzoic acid;
3-{[4-(hydroxymethyl)-2-methoxyphenoxy]methyl}benzoic acid;
5-phenyl[1,2,5]oxadiazolo[3,4-b]pyrazine;
2,3,9-trimethoxy-6a,12a-dihydrochromoeo[3,4-b]chromen-12(6H)-one;
4-[(4-chlorobenzyl)oxy]-3-methoxy-N-(2-methyl-5-[1,3]oxazolo[4,5-b]pyridin-2-ylphenyl)benzamide;
3-(1,3-benzothiazol-2-ylthio)propanoic acid;
3-{[(4-ethoxyphenoxy)acetyl]amino}benzoic acid;
N-[4-chloro-3-(trifluoromethyl)phenyl]-1-[(2,5-dimethoxyphenyl)sulfonyl]piperidine-4-carboxamide;
N-[3,5-bis(trifluoromethyl)phenyl]-2-[(5-nitro-1H-benzimidazol-2-yl)thio]acetamide;
5-[4-(4-tert-butylbenzoyl)piperazin-1-yl]-2-nitro-N-(pyridin-3-ylmethyl)aniline;
3-{[(2-chloro-4-(hydroxymethyl)phenoxy]methyl}benzoic acid;
{[(2-chlorophenyl)amino][(3-fluorobenzyl)thio]methylene}malononitrile;
{[(4-bromophenyl)amino][(4-fluorobenzyl)thio]methylene}malononitrile; and
1-[(1S,3R,5R,7S)-3-(3,4-dimethylphenyl)adamantan-1-yl]methanamine;
   a pharmaceutically acceptable salt thereof;
   and any combination thereof for use in regulating skin pigmentation.
   19. The compound of paragraph 17 selected from:
(3,5-di-t-butyl-4-hydroxybenzylidene)malononitrile;
2-amino-N-{5-[6-(dimethylamino)-9H-purin-9-yl]-4-hydroxy-2-(hydroxymethyl)tetrahydrofuran-3-yl}-3-(4-methoxyphenyl)propanamide;
1,3,6-trihydroxy-7-methoxy-2,8-bis(3-methylbut-2-en-1-yl)-9H-xanthen-9-one;
(3S,3aR,4R,6S,6aR,7S,8R,9bR)-6-acetoxy-4-(butyryloxy)-3,3a-dihydroxy-3,6,9-trimethyl-8-{[(2Z)-2-methylbut-2-enoyl]oxy}-2-oxo-2,3,3a,4,5,6,6a,7,8,9b-decahydroazuleno[4,5-b]furan-7-ylhexanoate;
(1E,5S)-6,6,6-trifluoro-5-hydroxy-1,5-diphenylhex-1-en-3-one;
2-(4-methoxyphenyl)-1,3-benzothiazole;
Ethyl-4-amino-2-{[(1E)-3-(4-methylphenyl)-3-oxoprop-1-en-1-yl]thio}pyrimidine-5-carboxylate;
3-[(5-chloro-1,3-benzothiazol-2-yl)thio]-5,5-dimethylcyclohex-2-en-1-one;
(2E)-3-[(5-methyl-1H-benzimidazol-2-yl)thio]-1-phenylprop-2-en-1-one;
11,12-dihydroxy-3-isobutyl-4,5,8-trimethyl-3,3a,4,6a,9,10,11,12-octahydro-1H-cycloundeca[d]isoindole-1,15(2H)-dione;
3-(hydroxymethyl)-6-isopropyl-5-[(2-methylbut-2-enoyl)oxy]-2-oxocyclohex-3-en-1-yl4-hydroxy-2-methylbut-2-enoate;
1,11,12-trihydroxy-2,16-dioxo-11,20-epoxypicras-3-en-15-yl2-hydroxy-2-methylbutanoate;
1-[3,5-bis(trifluoromethyl)phenyl]-3-[3-chloro-4-(isopropylsulfonyl)-2-thienyl]urea;
   a pharmaceutically acceptable salt thereof;
   and any combination thereof for use in regulating skin pigmentation.
   20. The compound of paragraph 17 selected from:
1,3,6-trihydroxy-7-methoxy-2,8-bis(3-methylbut-2-en-1-yl)-9H-xanthen-9-one;
(1E,5S)-6,6,6-trifluoro-5-hydroxy-1,5-diphenylhex-1-en-3-one;
3-(hydroxymethyl)-6-isopropyl-5-[(2-methylbut-2-enoyl)oxy]-2-oxocyclohex-3-en-1-yl-4-hydroxy-2-methylbut-2-enoate;
   a pharmaceutically acceptable salt thereof;
   and any combination thereof for use in regulating skin pigmentation.
   21. An assay for identifying a compound that modulate the expression or activity of Microphthalmia Transcription Factor (MITF), the method comprising:
      (i) contacting a cell with a test compound, wherein the cell comprises a reporter gene operably linked to a promoter of a direct transcriptional target of MITF, and wherein the cell is capable of producing MITF; and
      (ii) determining the amount of reporter gene expression after incubation with the test compound;
      wherein a change in the expression of the reporter gene relative to a control or reference indicates that the compound modulates the expression or activity of MITF.
   22. The assay of paragraph 21, wherein the test compound inhibits the expression or activity of MITF relative to a control or reference.
   23. The assay of paragraph 21 or 22, wherein the promoter is a promoter of TRPM1 gene.
   24. The assay of any of paragraphs 21-23, wherein the reporter gene is luciferase gene.
   25. The assay of any of paragraphs 21-24, wherein the cell endogenously produces MITF.
   26. The assay of any of paragraphs 21-25, wherein the cell is a melanocyte or from a melanoma cell line.
   27. The assay of any of paragraphs 21-26, wherein the test compound is incubated at a final concentration of from 0.01 nm to about 10 mM.
   28. The assay of any of paragraphs 21-27, wherein the test compound is tested at 2 or more different concentrations.
   29. The assay of paragraph 28, wherein the test compound is tested at 2 more or different concentrations in a 10-10,000 fold range.
   30. The assay of any of paragraphs 21-29, wherein the test compound is selected from the group consisting of small organic or inorganic molecules; saccharines; oligosaccharides; polysaccharides; biological macromolecules, e.g., peptides, proteins, and peptide analogs and derivatives; peptidomimetics; nucleic acids and nucleic acid analogs and derivatives (including but not limited to siRNAs, shRNAs, antisense RNAs, a ribozymes, and apatamers); an extract made from biological materials such as bacteria, plants, fungi, or animal cells; animal tissues; naturally occurring or synthetic compositions; and any combinations thereof.
   31. The assay of any of paragraphs 21-30, wherein the selected compound has a Log P value less than 7.
   32. The assay of any of paragraphs 21-31, wherein the selected compound exhibits dose-dependent modulation of MITF activity or expression.
   33. The assay of any of paragraphs 21-32, wherein the assay is a high throughput screening (HTS) assay.
   34. A compound selected by the assay of any of paragraphs 21-33.

### Examples

In order to carry out a successful MITF antagonist screen, the inventors utilized a TRPM1-luciferase reporter. The inventors had observed that the TRPM1 gene contains multiple MITF binding sites and exhibits an extremely robust upregulation in response to MITF transcriptional activation. Therefore, the TRPM1-luciferase reporter stably integrated into melanoma cells is effective for the screening assay. The use of melanoma cells, while less perfectly relevant than primary human melanocytes, afforded the advantages of speed and reproducibility for the screen. In addition, most human melanoma cell lines exhibit a baseline of cAMP signaling which is not dependent upon exogenously supplied Melanocyte Stimulating Hormone or cAMP agonists, thereby further contributing to the uniformity of the assay system.

In order to engineer a screen which broadly suppress MITF activity either by suppression of MITF expression or by inhibition of MITF activity, the inventors utilized a luciferase reporter containing the promoter of a strong direct transcriptional target of MITF, i.e., TRPM1 gene, the inventors have identified the active ingredients having the ability of inhibiting the expression or activation of Microphthalmia Transcription Factor (MITF) for medical or esthetic benefit. The inventors have previously identified the TRPM1 gene (also called “Melastatin”) as a direct target of MITF, which contains numerous MITF binding sites, and which directly responds in potent fashion to MITF overexpression (Miller A J, Du J, Rowan S, Hershey C L, Widlund H R, Fisher D E: Transcriptional regulation of the melanoma prognostic marker melastatin (TRPM1) by MITF in melanocytes and melanoma, Cancer Res 2004, 64:509-516). TRPM1 expression also correlates closely to MITF expression within human melanoma cell lines, as well as in clinical pigmented lesions (nevi and melanomas).

The TRPM1 promoter was ligated to the luciferase gene and inserted into a retroviral vector as well as a lentiviral vector. The lentiviral vector was used to infect primary human melanocytes, which then was subjected to a primary screen using the Broad Chemical Genetics core screening facility, and its natural products/extracts libraries. As backup, the retroviral vector was used to stably integrate into the genome of melanoma cells, which can be used for the identical small molecule screen. Screen optimization was carried out using forskolin and MSH as positive control small molecules (which upregulate cAMP and MITF expression (D'Orazio J A, Nobuhisa T, Cui R, Arya M, Spry M, Wakamatsu K, Igras V, Kunisada T, Granter S R, Nishimura E K, Ito S, Fisher D E: Topical drug rescue strategy and skin protection based on the role of Mc1r in UV-induced tanning, Nature 2006, 443:340-344)). Small molecules which suppress MSH induced TRPM1-luciferase without altering cell numbers or viability, represent initial “hits” in this screen. Negative control includes a constitutive luciferase vector within the same cells (eg driven by a constitutive promoter such as thymidine kinase promoter), to control for nonspecific inhibitors of basal transcriptional machinery-something which would otherwise be assessed in detail during secondary/tertiary analyses of candidate hits.

### Screen Design

The human melanoma cell line Skme15 was utilized due to the advantageous growth properties as well as the inventors' prior experience that Skme15 expresses a typical level of MITF protein. In addition, the inventors' prior experience has demonstrated that MITF levels correlate with expression of multiple pigmentation target genes of MITF, which strongly suggests that the transcriptional control of the pigmentation pathway by MITF remains intact within this cell line. Therefore, an integrated TRPM1-luciferase reporter was generated, and the subsequent cells were cloned twice (serially) to assure purity of the reporter cell population. Cells were maintained in drug selection to assure maintenance of the luciferase reporter (when drug was omitted, eventual loss of the luciferase signal was observed). Following re-cloning, distinct isolates were selected for further use on the basis of clonogenic growth on 384 well plates, as required for the small molecule screen. A positive control for suppression of TRPM1-luciferase signal utilized: a lentivirus encoding shRNA against MITF. This vector produced reproducible downregulation of MITF mRNA levels, as well as corresponding (>90%) reduction in luciferase measurements. The day-to-day variability in plating efficiency was analyzed, and found very high reproducibility in the line which was selected for further use in the screen.

The screen was carried out with a library from the Broad Institute comprising a mixture of natural products, known bioactives, and additional compounds (a total of about 13,000 compounds). The initial stages of data acquisition and analysis are summarized in Table 1. As indicated, “hits” were defined by suppression of TRPM1-luciferase activity, and comprised approximately 1% of screened molecules (about 130 compounds). Additional analyses/validation steps to further assess reproducibility, dose dependency, and potency/magnitude of the effects for candidate hits which suppress the reporter in this screen were carried out.

The first step in the analysis was to carry out a direct test of reproducibility. Compounds worthy of continued analyses needed to behave similarly upon repeat screening. Secondly, the ChemBank analytical tool at the Broad Institute was utilized. ChemBank is a database of results for small molecule screens, regardless of the precise nature of the screen, as long as they were carried out at the Broad Institute. The value in examining behavior of candidate hits on this dataset is that it will help the inventors rapidly determine whether the hits are unique to the screen, or whether they also “scored” in additional unrelated screens. From the hundreds of screens which are included within the ChemBank dataset, the inventors chose to retain candidate hits only if they scored on 3 or fewer additional screens listed on ChemBank (out of several hundred screens, a number which is constantly growing). The third validation criterion was behavior on a dose titration analysis.

Candidate hits were acceptable only if they exhibited dose-dependent suppression of the TRPM1-luciferase signal. Although the shape of the dose-response can vary among hits, the inventors chose to include all candidates which produce dose-dependent effects. The fourth criterion which was determined is the IC50 (described formally as qAC50) which represents the half-maximal suppression dose. Most of the candidate hits resided in the high nanomolar to low micromolar range by this criterion (all of these validation data are listed in Table 1). The fifth criterion was maximal suppression (at any dose), which represented the magnitude of suppression which was attainable regardless of the dose required to achieve it. This is a valuable criterion because some highly potent inhibitors (based on IC50) can yield only weak maximal suppression, whereas other less potent inhibitors (based on IC50) can produce significantly greater suppression, even at doses below their IC50. The sixth criterion to be determined is the Log P partition coefficient, which is a predictor of lipophilicity. For this, the inventors have used the method of Ghose and Crippen to calculate the predicted octanol-water partition coefficient (J. Comp. Chem. 9, 80(1988)). While this is only an estimate of potential skin penetration, the analysis was carried out because it helps in prioritizing likelihood of topical effectiveness, in theory. The seventh criterion was an analysis of toxicity utilizing a fluorescence viability probe (CellTiter-Fluo™ Cell Viability Assay (Promega)). Interpretation of this endpoint is complicated by the fact that it represents a composite of the variation between drug-treated and control divided by the standard error The results shown in Table 1 indicate 34 candidate molecules which satisfied all of the criteria outlined above. Specifically, these compounds exhibited reproducible suppression of TRPM1-luciferase as well as dose-response behaviors. Most of their potencies ranged slightly below or above 1 uM. While the lipophilicity scores remain imperfect predictors of skin penetration capability. However, numbers of >3 can be more likely to penetrate. As seen from data in Table 1, a significant number of hits fit that criterion.

TABLE I

| Compound | AlogP | CMPD_Plate_MAP | Z-Score | Reproducibility |
|---|---|---|---|---|
| 1-[(1S,3R,5R,7S)-3-(3,4- | 4.0369999999999999218 | H-COMA-003-1 | −10.8562 | 0.999048825 |
| dimethylphenyl) adamantan-1- | | | | |
| yl]methanamine | | | | |
| 5-chloro-7-[(2-chloro-6- | 5.7009999999999996234 | H-KINA-029-1 | −9.32669 | 0.989483808 |
| fluorophenyl) (pyridin-2-ylamino) | | | | |
| methyl]quinolin-8-ol | | | | |
| (1S,2R,3S,6S)-1-benzoyl-6-tert- | 4.1920000000000001705 | H-COMA-007-1 | −11.0899 | 0.998865691 |
| butylspiro[2.5]octane-1,2- | | | | |
| dicarbonitrile | | | | |
| N-[(4-chlorophenyl)(2-hydroxy-1- | 5.5480000000000000426 | H-KINA-018-1 | −11.9861 | 0.989037581 |
| naphthyl)methyl]-3- | | | | |
| methoxybenzamide | | | | |
| Ethyl-1-benzyl-5-[3-(tert-butylamino)- | 4.2690000000000001279 | H-COMA-005-1 | −7.84416 | 0.963051252 |
| 2-hydroxypropoxy]-2-methyl-1H-indole- | | | | |
| 3-carboxylate | | | | |
| 5-chloro-7-[(2,5-dimethylphenyl) | 5.80400000000000027 | H-KINA-021-1 | −8.46137 | 0.983811186 |
| (pyridin-2-ylamino) methyl]quinolin- | | | | |
| 8-ol | | | | |
| N-[5-(2,3-dihydro-1,4- | 1.3639999999999998792 | H-KINA-020-1 | −10.0065 | 0.998701418 |
| benzodioxin-6-yl)-2-methyl-3- | | | | |
| furoyl]glycine | | | | |
| [3-(1,3-benzothiazol-2- | 3.3039999999999998259 | H-KINA-008-1 | −11.9101 | 0.997997864 |
| yl)phenoxy]acetic acid | | | | |
| N-(2-fluorophenyl)-2-{[5-(3- | 3.5400000000000000355 | H-KINA-020-1 | −10.7661 | 0.999967366 |
| methylphenyl)-1,3,4-oxadiazol-2- | | | | |
| yl]thio}acetamide | | | | |
| N-[4-(6-methyl-1,3-benzothiazol-2- | 4.2379999999999995453 | H-KINA-023-1 | −9.3543 | 0.99452731 |
| yl)phenyl]-2-[(5-methyl-1,3,4-thiadiazol- | | | | |
| 2-yl)thio]acetamide | | | | |
| 2-(2-bromo-4-tert-butylphenoxy)-N- | 4.4759999999999999787 | H-KINA-010-1 | −12.0562 | 0.996556144 |
| (2-methoxy-5-nitrophenyl) | | | | |
| acetamide | | | | |
| N-phenyl-3-[(4-phenyl-1,3-thiazol-2- | 4.2000000000000001776 | H-KINA-016-1 | −10.8255 | 0.998902877 |
| yl)thio]propanamide | | | | |
| Ethyl [3-(1,3-benzothiazol-2- | 3.8780000000000001137 | H-KINA-021-1 | −9.66315 | 0.996892511 |
| yl)phenoxy]acetate | | | | |
| 2-methyl-3-[(4-methylpiperidin-1- | 2.9319999999999999396 | H-COMA-011-1 | −9.92374 | 0.999280956 |
| yl)carbonyl]naphtho[2,3-b]furan-4,9- | | | | |
| dione | | | | |
| N-(3-phenylpropyl)-10H- | 5.3780000000000001137 | H-COMA-005-1 | −9.49491 | 0.996850399 |
| phenothiazine-10-carboxamide | | | | |
| 5-(4-chlorophenyl)-2-pyrrolidin-1-yl- | 3.4779999999999997584 | H-COMA-003-1 | −9.96123 | 0.999451704 |
| 6H-1,3,4-thiadiazine | | | | |
| 7-(4-fluorophenyl)-1-(2-furylmethyl)- | 3.5049999999999998934 | H-KINA-032-1 | −8.79137 | 0.989155549 |
| 5,5-bis(trifluoromethyl)-5,8- | | | | |
| dihydropyrimido[4,5-d]pyrimidine- | | | | |
| 2,4(1H,3H)-dione | | | | |
| 3-{[(2-bromo-4-methylphenoxy) | 3.3140000000000000568 | H-KINA-030-1 | −10.0837 | 0.992087627 |
| acetyl]amino}benzoic acid | | | | |
| 3{[4-(hydroxymethyl)-2- | 2.4060000000000001386 | H-KINA-014-1 | −10.9296 | 0.99982555 |
| methoxyphenoxy] methyl} benzoic | | | | |
| acid | | | | |
| 5-phenyl[1,2,5]oxadiazolo[3,4- | 1.4359999999999999432 | H-COMA-006-1 | −9.98917 | 0.998804969 |
| b]pyrazine | | | | |
| 2,3,9-trimethoxy-6a,12a- | 2.7640000000000002345 | H-BIOA-005-1 | −9.24474 | 0.994367707 |
| dihydrochromoeo[3,4-b]chromen- | | | | |
| 12(6H)-one | | | | |
| 4-[(4-chlorobenzyl)oxy]-3-methoxy-N-(2- | 5.8109999999999999432 | H-KINA-003-1 | −9.25626 | 0.973443672 |
| methyl-5-[1,3]oxazolo[4,5-b]pyridin-2- | | | | |
| ylphenyl)benzamide | | | | |
| 3-(1,3-benzothiazol-2-ylthio) | 2.7259999999999999787 | H-KINA-032-1 | −9.79048 | 0.998368331 |
| propanoic acid | | | | |
| 3-{[(4-ethoxyphenoxy) acetyl] | 2.4119999999999999218 | H-KINA-019-1 | −9.90022 | 0.99879666 |
| amino} benzoic acid | | | | |
| 4-[(4-chlorobenzyl)oxy]-3-methoxy- | 5.8109999999999999432 | H-KINA-003-1 | −9.25626 | 0.973443672 |
| N-(2-methyl-5-[1,3]oxazolo[4,5- | | | | |
| b]pyridin-2-ylphenyl)benzamide | | | | |
| 3-(1,3-benzothiazol-2-ylthio) | 2.7259999999999999787 | H-KINA-032-1 | −9.79048 | 0.998368331 |
| propanoic acid | | | | |
| 3-{[(4-ethoxyphenoxy) acetyl] | 2.4119999999999999218 | H-KINA-019-1 | −9.90022 | 0.99879666 |
| amino} benzoic acid | | | | |
| N-[4-chloro-3-(trifluoromethyl) | 3.777000000000000135 | H-KINA-009-1 | −9.87118 | 0.999636801 |
| phenyl]-1-[(2,5-dimethoxyphenyl) | | | | |
| sulfonyl]piperidine-4-carboxamide | | | | |
| N-[3,5-bis(trifluoromethyl) phenyl]-2- | 4.8440000000000003055 | H-KINA-006-1 | −10.0284 | 0.999098189 |
| [(5-nitro-1H-benzimidazol-2-yl) thio] | | | | |
| acetamide | | | | |
| 5-[4-(4-tert-butylbenzoyl) | 4.4089999999999998082 | H-KINA-013-1 | −9.58847 | 0.995934272 |
| piperazin-1-yl]-2-nitro-N- | | | | |
| (pyridin-3-ylmethyl) aniline | | | | |
| 3-{[2-chloro-4-(hydroxymethyl) | 3.0859999999999998543 | H-KINA-013-1 | −9.90876 | 0.999215485 |
| phenoxy] methyl} benzoic acid | | | | |
| {[(2-chlorophenyl)amino][(3- | 5.0330000000000003624 | H-COMA-011-1 | −10.3064 | 0.99873536 |
| fluorobenzyl)thio] methylene} | | | | |
| malononitrile | | | | |
| {[(4-bromophenyl) amino][(4- | 5.1169999999999999929 | H-COMA-011-1 | −9.46271 | 0.996715021 |
| fluorobenzyl) thio] methylene} | | | | |
| malononitrile | | | | |
| 1-[(1S,3R,5R,7S)-3-(3,4- | 4.0369999999999999218 | H-COMA-003-1 | −10.8562 | 0.999048825 |
| dimethylphenyl) adamantan-1- | | | | |
| yl]methanamine | | | | |

| Compound | Fluorescence | Maximal Activity | Conc. At Maximal Activity | qAC50 |
|---|---|---|---|---|
| 1-[(1S,3R,5R,7S)-3-(3,4- | −1.01495 | −149 | 6.00E−06 | 2.48E−06 |
| dimethylphenyl) adamantan-1- | | | | |
| yl]methanamine | | | | |
| 5-chloro-7-[(2-chloro-6- | −2.226 | −283.5 | 1.00E−05 | 1.86E−07 |
| fluorophenyl) (pyridin-2-ylamino) | | | | |
| methyl]quinolin-8-ol | | | | |
| (1S,2R,3S,6S)-1-benzoyl-6-tert- | 0.48235 | −274.1 | 1.95E−05 | 5.69E−06 |
| butylspiro[2.5]octane-1,2- | | | | |
| dicarbonitrile | | | | |
| N-[(4-chlorophenyl)(2-hydroxy-1- | −0.1221 | −266.4 | 1.20E−05 | 9.56E−07 |
| naphthyl)methyl]-3- | | | | |
| methoxybenzamide | | | | |
| Ethyl-1-benzyl-5-[3-(tert-butylamino)- | −2.51945 | −134 | 1.35E−05 | 3.27E−06 |
| 2-hydroxypropoxy]-2-methyl-1H-indole- | | | | |
| 3-carboxylate | | | | |
| 5-chloro-7-[(2,5-dimethylphenyl) | −0.88935 | −185.8 | 1.10E−05 | 1.09E−07 |
| (pyridin-2-ylamino) methyl]quinolin- | | | | |
| 8-ol | | | | |
| N-[5-(2,3-dihydro-1,4- | −0.0206 | −143.2 | 1.60E−05 | 6.13E−06 |
| benzodioxin-6-yl)-2-methyl-3- | | | | |
| furoyl]glycine | | | | |
| [3-(1,3-benzothiazol-2- | −0.0206 | −281.2 | 1.60E−05 | 9.21E−07 |
| yl)phenoxy]acetic acid | | | | |
| N-(2-fluorophenyl)-2-{[5-(3- | −0.0206 | −274.8 | 1.35E−05 | 4.02E−06 |
| methylphenyl)-1,3,4-oxadiazol-2- | | | | |
| yl]thio}acetamide | | | | |
| N-[4-(6-methyl-1,3-benzothiazol-2- | 1.03175 | 299.1 | 1.80E−07 | 1.91E−06 |
| yl)phenyl]-2-[(5-methyl-1,3,4-thiadiazol- | | | | |
| 2-yl)thio]acetamide | | | | |
| 2-(2-bromo-4-tert-butylphenoxy)-N- | −1.6406 | −261.3 | 1.00E−05 | 2.92E−07 |
| (2-methoxy-5-nitrophenyl) | | | | |
| acetamide | | | | |
| N-phenyl-3-[(4-phenyl-1,3-thiazol-2- | −0.2442 | −486 | 1.35E−05 | 3.10E−07 |
| yl)thio]propanamide | | | | |
| Ethyl [3-(1,3-benzothiazol-2- | −0.0206 | −241.3 | 1.60E−05 | 1.89E−06 |
| yl)phenoxy]acetate | | | | |
| 2-methyl-3-[(4-methylpiperidin-1- | 0.15755 | −114 | 1.80E−05 | 2.15E−06 |
| yl)carbonyl]naphtho[2,3-b]furan-4,9- | | | | |
| dione | | | | |
| N-(3-phenylpropyl)-10H- | −4.12765 | −188.5 | 1.80E−05 | 9.33E−06 |
| phenothiazine-10-carboxamide | | | | |
| 5-(4-chlorophenyl)-2-pyrrolidin-1-yl- | −0.2442 | −267.4 | 1.80E−05 | 1.57E−06 |
| 6H-1,3,4-thiadiazine | | | | |
| 7-(4-fluorophenyl)-1-(2-furylmethyl)- | −2.0352 | −454.8 | 9.00E−06 | 1.09E−06 |
| 5,5-bis(trifluoromethyl)-5,8- | | | | |
| dihydropyrimido[4,5-d]pyrimidine- | | | | |
| 2,4(1H,3H)-dione | | | | |
| 3-{[(2-bromo-4-methylphenoxy) | −1.5092 | −335.7 | 1.20E−05 | 3.71E−07 |
| acetyl]amino}benzoic acid | | | | |
| 3{[4-(hydroxymethyl)-2- | −1.6406 | −462.9 | 1.60E−05 | 6.45E−07 |
| methoxyphenoxy] methyl} benzoic | | | | |
| acid | | | | |
| 5-phenyl[1,2,5]oxadiazolo[3,4- | −0.0206 | −328.9 | 3.00E−05 | 1.44E−06 |
| b]pyrazine | | | | |
| 2,3,9-trimethoxy-6a,12a- | −4.2731 | −320.4 | 1.20E−05 | 5.38E−06 |
| dihydrochromoeo[3,4-b]chromen- | | | | |
| 12(6H)-one | | | | |
| 4-[(4-chlorobenzyl)oxy]-3-methoxy-N-(2- | | −150.5 | 1.10E−06 | 7.40E−07 |
| methyl-5-[1,3]oxazolo[4,5-b]pyridin-2- | | | | |
| ylphenyl)benzamide | | | | |
| 3-(1,3-benzothiazol-2-ylthio) | −1.5092 | −321.2 | 1.80E−05 | 1.54E−06 |
| propanoic acid | | | | |
| 3-{[(4-ethoxyphenoxy) acetyl] | −1.5092 | −206.5 | 1.50E−05 | 1.55E−06 |
| amino} benzoic acid | | | | |
| 4-[(4-chlorobenzyl)oxy]-3-methoxy- | | −150.5 | 1.10E−06 | 7.40E−07 |
| N-(2-methyl-5-[1,3]oxazolo[4,5- | | | | |
| b]pyridin-2-ylphenyl)benzamide | | | | |
| 3-(1,3-benzothiazol-2-ylthio) | −1.5092 | −321.2 | 1.80E−05 | 1.54E−06 |
| propanoic acid | | | | |
| 3-{[(4-ethoxyphenoxy) acetyl] | −1.5092 | −206.5 | 1.50E−05 | 1.55E−06 |
| amino} benzoic acid | | | | |
| N-[4-chloro-3-(trifluoromethyl) | −0.2442 | −208.8 | 1.00E−05 | 1.65E−06 |
| phenyl]-1-[(2,5-dimethoxyphenyl) | | | | |
| sulfonyl]piperidine-4-carboxamide | | | | |
| N-[3,5-bis(trifluoromethyl) phenyl]-2- | | −362.8 | 4.20E−06 | 4.84E−07 |
| [(5-nitro-1H-benzimidazol-2-yl) thio] | | | | |
| acetamide | | | | |
| 5-[4-(4-tert-butylbenzoyl) | −0.88935 | −445.2 | 1.00E−05 | 4.55E−06 |
| piperazin-1-yl]-2-nitro-N- | | | | |
| (pyridin-3-ylmethyl) aniline | | | | |
| 3-{[2-chloro-4-(hydroxymethyl) | −1.5092 | −347 | 4.20E−06 | 2.72E−07 |
| phenoxy] methyl} benzoic acid | | | | |
| {[(2-chlorophenyl)amino][(3- | −1.5092 | −510 | 4.60E−06 | 1.77E−06 |
| fluorobenzyl)thio] methylene} | | | | |
| malononitrile | | | | |
| {[(4-bromophenyl) amino][(4- | −1.5092 | −403.8 | 1.95E−06 | 2.30E−07 |
| fluorobenzyl) thio] methylene} | | | | |
| malononitrile | | | | |
| 1-[(1S,3R,5R,7S)-3-(3,4- | −1.01495 | −149 | 6.00E−06 | 2.48E−06 |
| dimethylphenyl) adamantan-1- | | | | |
| yl]methanamine | | | | |

The inventors have developed a core facility for small-molecule screening at the Cutaneous Biology Research Center (CBRC). The inventors currently have automated equipment, which allows them to: 1) plate cells in 96- and 384-well format; 2) pin-transfer small-molecule compounds from chemical libraries into 96- and 384-well tissue culture plates; 3) read luminescence, fluorescence and absorbance in multi-well plates. The equipment installed provides unique possibility to perform complete process for high throughput screening.

The luciferase gene was cloned under the transcriptional control of MITF-binding sites to respond to negative or positive regulation of MITF expression and/or activity. Using this reporter system in SK-MELS cells, a chemical library of ˜2000 compounds representing bioactive compounds, commercially available and natural products was screened. The screening was repeated two independent times in duplicated wells at the Broad Institute using their automated equipment. Hits were selected based on 4 times suppression of the reporter and reproducibility between the two independent screens. The data analysis revealed 18 compounds being highly reproducible between the screens performed at the Broad Institute. When the inventors repeated this screening at CBRC, they aimed to reproduce those hits. The same experimental settings and the same chemical library were used. Below is a detailed protocol for the assay performed. Data analysis of the screening applying the same criteria of 4 times suppression of the reporter when compared to DMSO wells demonstrated that 13 of the 18 compounds selected as hits from the Broad screen scored as hits at CBRC, which validate the equipment and workflow.

### Assay Protocol (Equipment: Well Mate, Metrix Inc.; Rapid Plate, Caliper Inc; Victor X3, Perkin Elmer Inc.)

1. On Day 1 plate 5000 SK-MELS-Luc cells per well in 25 μl medium (DMEM, 10% FBS, 1× PenStrep).
2. On Day 2 pin transfer 100 nl of chemical library (10 mM library concentration).
3. On Day 3, 24 h after the pin transfer, add 25 μl SteadyLite Plus (Perkin Elmer) reagent; vortex for 2 min at 60 rpm. Measure luminescence for 1.5 seconds.

1. A method for regulating skin pigmentation comprising: administering to a subject in need thereof, a pharmaceutical composition comprising a therapeutic agent inhibiting the expression or activation of Microphthalmia Transcription Factor (MITF).

2. The method of claim 1, wherein the therapeutic agent is selected from the group consisting of:
1-[(1S,3R,5R,7S)-3-(3,4-dimethylphenyl)adamantan-1-yl]methanamine;
5-chloro-7-[(2-chloro-6-fluorophenyl)(pyridin-2-ylamino)methyl]quinolin-8-ol;
(1S,2R,3S,6S)-1-benzoyl-6-tert-butylspiro[2.5]octane-1,2-dicarbonitrile;
N-[(4-chlorophenyl)(2-hydroxy-1-naphthyl)methyl]-3-methoxybenzamide;
Ethyl-1-benzyl-5-[3-(tert-butylamino)-2-hydroxypropoxy]-2-methyl-1H-indole-3-carboxylate;
5-chloro-7-[(2,5-dimethylphenyl)(pyridin-2-ylamino)methyl]quinolin-8-ol;
N-[5-(2,3-dihydro-1,4-benzodioxin-6-yl)-2-methyl-3-furoyl]glycine;
[3-(1,3-benzothiazol-2-yl)phenoxy]acetic acid;
N-(2-fluorophenyl)-2-{[5-(3-methylphenyl)-1,3,4-oxadiazol-2-yl]thio}acetamide;
N-[4-(6-methyl-1,3-benzothiazol-2-yl)phenyl]-2-[(5-methyl-1,3,4-thiadiazol-2-yl)thio]acetamide;
2-(2-bromo-4-tert-butylphenoxy)-N-(2-methoxy-5-nitrophenyl)acetamide;
Methyl-5-{[(4-fluoro-2-methylphenyl)sulfonyl](isonicotinoyl)amino}-2-methyl-1-benzofuran-3-carboxylate;
1-(4-chlorobenzyl)-N-(pyridin-3-ylmethyl)-1H-pyrazolo[3,4-d]pyrimidin-4-amine;
2-{[5-(4-chlorophenyl)-4-(3-methylphenyl)-4H-1,2,4-triazol-3-yl]thio}-N-(3-methoxyphenyl)acetamide;
N-phenyl-3-[(4-phenyl-1,3-thiazol-2-yl)thio]propanamide;
Ethyl-[3-(1,3-benzothiazol-2-yl)phenoxy]acetate;
2-methyl-3-[(4-methylpiperidin-1-yl)carbonyl]naphtho[2,3-b]furan-4,9-dione;
N-(3-phenylpropyl)-10H-phenothiazine-10-carboxamide;
5-(4-chlorophenyl)-2-pyrrolidin-1-yl-6H-1,3,4-thiadiazine;
7-(4-fluorophenyl)-1-(2-furylmethyl)-5,5-bis(trifluoromethyl)-5,8-dihydropyrimido[4,5-d]pyrimidine-2,4(1H,3H)-dione;
3-{[(2-bromo-4-methylphenoxy)acetyl]amino}benzoic acid;
3-{[4-(hydroxymethyl)-2-methoxyphenoxy]methyl}benzoic acid;
5-phenyl[1,2,5]oxadiazolo[3,4-b]pyrazine;
2,3,9-trimethoxy-6a, 12a-dihydrochromoeo[3,4-b]chromen-12(6H)-one;
4-[(4-chlorobenzyl)oxy]-3-methoxy-N-(2-methyl-5-[1,3]oxazolo[4,5-b]pyridin-2-ylphenyl)benzamide;
3-(1,3-benzothiazol-2-ylthio)propanoic acid;
3-{[(4-ethoxyphenoxy)acetyl]amino}benzoic acid;
N-[4-chloro-3-(trifluoromethyl)phenyl]-1-[(2,5-dimethoxyphenyl)sulfonyl]piperidine-4-carboxamide;
N-[3,5-bis(trifluoromethyl)phenyl]-2-[(5-nitro-1H-benzimidazol-2-yl)thio]acetamide;
5-[4-(4-tert-butylbenzoyl)piperazin-1-yl]-2-nitro-N-(pyridin-3-ylmethyl)aniline;
3-{[2-chloro-4-(hydroxymethyl)phenoxy]methyl}benzoic acid;
{[(2-chlorophenyl)amino][(3-fluorobenzyl)thio]methylene}malononitrile; and
{[(4-bromophenyl)amino][(4-fluorobenzyl)thio]methylene}malononitrile;
(3,5-di-t-butyl-4-hydroxybenzylidene)malononitrile;
2-amino-N-{5-[6-(dimethylamino)-9H-purin-9-yl]-4-hydroxy-2-(hydroxymethyl)tetrahydrofuran-3-yl}-3-(4-methoxyphenyl)propanamide;
1,3,6-trihydroxy-7-methoxy-2,8-bis(3-methylbut-2-en-1-yl)-9H-xanthen-9-one;
(3S,3aR,4R,6S,6aR,7S,8R,9bR)-6-acetoxy-4-(butyryloxy)-3,3a-dihydroxy-3,6,9-trimethyl-8-{[(2Z)-2-methylbut-2-enoyl]oxy}-2-oxo-2,3,3a,4,5,6,6a,7,8,9b-decahydroazuleno[4,5-b]furan-7-ylhexanoate;
(1E,5S)-6,6,6-trifluoro-5-hydroxy-1,5-diphenylhex-1-en-3-one;
2-(4-methoxyphenyl)-1,3-benzothiazole;
Ethyl-4-amino-2-{[(1E)-3-(4-methylphenyl)-3-oxoprop-1-en-1-yl]thio}pyrimidine-5-carboxylate;
3-[(5-chloro-1,3-benzothiazol-2-yl)thio]-5,5-dimethylcyclohex-2-en-1-one;
(2E)-3-[(5-methyl-1H-benzimidazol-2-yl)thio]-1-phenylprop-2-en-1-one;
11,12-dihydroxy-3-isobutyl-4,5,8-trimethyl-3,3a,4,6a,9,10,11,12-octahydro-1H-cycloundeca[d]isoindole-1,15(2H)-dione;
3-(hydroxymethyl)-6-isopropyl-5-[(2-methylbut-2-enoyl)oxy]-2-oxocyclohex-3-en-1-yl4-hydroxy-2-methylbut-2-enoate;
1,11,12-trihydroxy-2,16-dioxo-11,20-epoxypicras-3-en-15-yl2-hydroxy-2-methylbutanoate;
1-[3,5-bis(trifluoromethyl)phenyl]-3-[3-chloro-4-(isopropylsulfonyl)-2-thienyl]urea;
a pharmaceutical salt thereof;
and any combination thereof.

3. The method of claim 2, wherein the therapeutic agent is selected from the group consisting of:
1-[(1S,3R,5R,7S)-3-(3,4-dimethylphenyl)adamantan-1-yl]methanamine;
5-chloro-7-[(2-chloro-6-fluorophenyl)(pyridin-2-ylamino)methyl]quinolin-8-ol;
(1S,2R,3S,6S)-1-benzoyl-6-tert-butylspiro[2.5]octane-1,2-dicarbonitrile;
N-[(4-chlorophenyl)(2-hydroxy-1-naphthyl)methyl]-3-methoxybenzamide;
Ethyl-1-benzyl-5-[3-(tert-butylamino)-2-hydroxypropoxy]-2-methyl-1H-indole-3-carboxylate;
5-chloro-7-[(2,5-dimethylphenyl)(pyridin-2-ylamino)methyl]quinolin-8-ol;
N-[5-(2,3-dihydro-1,4-benzodioxin-6-yl)-2-methyl-3-furoyl]glycine;
[3-(1,3-benzothiazol-2-yl)phenoxy]acetic acid;
N-(2-fluorophenyl)-2-{[5-(3-methylphenyl)-1,3,4-oxadiazol-2-yl]thio}acetamide;
N-[4-(6-methyl-1,3-benzothiazol-2-yl)phenyl]-2-[(5-methyl-1,3,4-thiadiazol-2-yl)thio]acetamide;
2-(2-bromo-4-tert-butylphenoxy)-N-(2-methoxy-5-nitrophenyl)acetamide;
Methyl-5-{[(4-fluoro-2-methylphenyl)sulfonyl](isonicotinoyl)amino}-2-methyl-1-benzofuran-3-carboxylate;
1-(4-chlorobenzyl)-N-(pyridin-3-ylmethyl)-1H-pyrazolo[3,4-d]pyrimidin-4-amine;
2-{[5-(4-chlorophenyl)-4-(3-methylphenyl)-4H-1,2,4-triazol-3-yl]thio}-N-(3-methoxyphenyl)acetamide;
N-phenyl-3-[(4-phenyl-1,3-thiazol-2-yl)thio]propan amide;
Ethyl-[3-(1,3-benzothiazol-2-yl)phenoxy]acetate;
2-methyl-3-[(4-methylpiperidin-1-yl)carbonyl]naphtho[2,3-b]furan-4,9-dione;
N-(3-phenylpropyl)-10H-phenothiazine-10-carboxamide;
5-(4-chlorophenyl)-2-pyrrolidin-1-yl-6H-1,3,4-thiadiazine;
7-(4-fluorophenyl)-1-(2-furylmethyl)-5,5-bis(trifluoromethyl)-5,8-dihydropyrimido[4,5-d]pyrimidine-2,4(1H,3H)-dione;
3-{[(2-bromo-4-methylphenoxy)acetyl]amino}benzoic acid;
3-{[4-(hydroxymethyl)-2-methoxyphenoxy]methyl}benzoic acid;
5-phenyl[1,2,5]oxadiazolo[3,4-b]pyrazine;
2,3,9-trimethoxy-6a,12a-dihydrochromoeo[3,4-b]chromen-12(6H)-one;
4-[(4-chlorobenzyl)oxy]-3-methoxy-N-(2-methyl-5-[1,3]oxazolo[4,5-b]pyridin-2-ylphenyl)benzamide;
3-(1,3-benzothiazol-2-ylthio)propanoic acid;
3-{[(4-ethoxyphenoxy)acetyl]amino}benzoic acid;
N-[4-chloro-3-(trifluoromethyl)phenyl]-1-[(2,5-dimethoxyphenyl)sulfonyl]piperidine-4-carboxamide;
N-[3,5-bis(trifluoromethyl)phenyl]-2-[(5-nitro-1H-benzimidazol-2-yl)thio]acetamide;
5-[4-(4-tert-butylbenzoyl)piperazin-1-yl]-2-nitro-N-(pyridin-3-ylmethyl)aniline;
3-{[2-chloro-4-(hydroxymethyl)phenoxy]methyl}benzoic acid;
{[(2-chlorophenyl)amino][(3-fluorobenzyl)thio]methylene}malononitrile; and
{[(4-bromophenyl)amino][(4-fluorobenzyl)thio]methylene}malononitrile;
a pharmaceutically acceptable salt thereof;
and any combination thereof.

4. The method of claim 2, wherein the therapeutic agent is selected from the group consisting of:
(3,5-di-t-butyl-4-hydroxybenzylidene)malononitrile;
2-amino-N-{5-[6-(dimethylamino)-9H-purin-9-yl]-4-hydroxy-2-(hydroxymethyl)tetrahydrofuran-3-yl}-3-(4-methoxyphenyl)propanamide;
1,3,6-trihydroxy-7-methoxy-2,8-bis(3-methylbut-2-en-1-yl)-9H-xanthen-9-one;
(3S,3aR,4R,6S,6aR,7S,8R,9bR)-6-acetoxy-4-(butyryloxy)-3,3a-dihydroxy-3,6,9-trimethyl-8-{[(2Z)-2-methylbut-2-enoyl]oxy}-2-oxo-2,3,3a,4,5,6,6a,7,8,9b-decahydroazuleno[4,5-b]furan-7-ylhexanoate;
(1E,5S)-6,6,6-trifluoro-5-hydroxy-1,5-diphenylhex-1-en-3-one;
2-(4-methoxyphenyl)-1,3-benzothiazole;
Ethyl-4-amino-2-{[(1E)-3-(4-methylphenyl)-3-oxoprop-1-en-1-yl]thio}pyrimidine-5-carboxylate;
3-[(5-chloro-1,3-benzothiazol-2-yl)thio]-5,5-dimethylcyclohex-2-en-1-one;
(2E)-3-[(5-methyl-1H-benzimidazol-2-yl)thio]-1-phenylprop-2-en-1-one;
11,12-dihydroxy-3-isobutyl-4,5,8-trimethyl-3,3a,4,6a,9,10,11,12-octahydro-1H-cycloundeca[d]isoindole-1,15(2H)-dione;
3-(hydroxymethyl)-6-isopropyl-5-[(2-methylbut-2-enoyl)oxy]-2-oxocyclohex-3-en-1-yl4-hydroxy-2-methylbut-2-enoate;
1,11,12-trihydroxy-2,16-dioxo-11,20-epoxypicras-3-en-15-yl2-hydroxy-2-methylbutanoate;
1-[3,5-bis(trifluoromethyl)phenyl]-3-[3-chloro-4-(isopropylsulfonyl)-2-thienyl]urea;
a pharmaceutically acceptable salt thereof;
and any combination thereof.

5. The method of claim 2, wherein the therapeutic agent is selected from the group consisting of:
1,3,6-trihydroxy-7-methoxy-2,8-bis(3-methylbut-2-en-1-yl)-9H-xanthen-9-one;
(1E,5S)-6,6,6-trifluoro-5-hydroxy-1,5-diphenylhex-1-en-3-one;
3-(hydroxymethyl)-6-isopropyl-5-[(2-methylbut-2-enoyl)oxy]-2-oxocyclohex-3-en-1-yl-4-hydroxy-2-methylbut-2-enoate;
a pharmaceutically acceptable salt thereof;
and any combination thereof.

6. The method of claim 1, wherein pharmaceutical composition comprises an additional therapeutic agent.

7. The method of claim 1, wherein the pharmaceutical composition comprises 0.001%-99% of the therapeutic agent, inhibiting the expression or activation of MITF.

8. The method of claim 1, wherein the pharmaceutical composition is formulated as a topical formulation, a parenteral formulation, or an oral formulation.

9. (canceled)

10. (canceled)

11. (canceled)

12. A pharmaceutical composition comprising:
1-[(1S,3R,5R,7S)-3-(3,4-dimethylphenyl)adamantan-1-yl]methanamine;
5-chloro-7-[(2-chloro-6-fluorophenyl)(pyridin-2-ylamino)methyl]quinolin-8-ol;
(1S,2R,3S,6S)-1-benzoyl-6-tert-butylspiro[2.5]octane-1,2-dicarbonitrile;
N-[(4-chlorophenyl)(2-hydroxy-1-naphthyl)methyl]-3-methoxybenzamide;
Ethyl-1-benzyl-5-[3-(tert-butylamino)-2-hydroxypropoxy]-2-methyl-1H-indole-3-carboxylate;
5-chloro-7-[(2,5-dimethylphenyl)(pyridin-2-ylamino)methyl]quinolin-8-ol;
N-[5-(2,3-dihydro-1,4-benzodioxin-6-yl)-2-methyl-3-furoyl]glycine;
[3-(1,3-benzothiazol-2-yl)phenoxy]acetic acid;
N-(2-fluorophenyl)-2-{[5-(3-methylphenyl)-1,3,4-oxadiazol-2-yl]thio}acetamide;
N-[4-(6-methyl-1,3-benzothiazol-2-yl)phenyl]-2-[(5-methyl-1,3,4-thiadiazol-2-yl)thio]acetamide;
2-(2-bromo-4-tert-butylphenoxy)-N-(2-methoxy-5-nitrophenyl)acetamide;
Methyl-5-{[(4-fluoro-2-methylphenyl)sulfonyl](isonicotinoyl)amino}-2-methyl-1-benzofuran-3-carboxylate;
1-(4-chlorobenzyl)-N-(pyridin-3-ylmethyl)-1H-pyrazolo[3,4-d]pyrimidin-4-amine;
2-{[5-(4-chlorophenyl)-4-(3-methylphenyl)-4H-1,2,4-triazol-3-yl]thio}-N-(3-methoxyphenyl)acetamide;
N-phenyl-3-[(4-phenyl-1,3-thiazol-2-yl)thio]propanamide;
Ethyl-[3-(1,3-benzothiazol-2-yl)phenoxy]acetate;
2-methyl-3-[(4-methylpiperidin-1-yl)carbonyl]naphtho[2,3-b]furan-4,9-dione;
N-(3-phenylpropyl)-10H-phenothiazine-10-carboxamide;
5-(4-chlorophenyl)-2-pyrrolidin-1-yl-6H-1,3,4-thiadiazine;
7-(4-fluorophenyl)-1-(2-furylmethyl)-5,5-bis(trifluoromethyl)-5,8-dihydropyrimido[4,5-d]pyrimidine-2,4(1H,3H)-dione;
3-{[(2-bromo-4-methylphenoxy)acetyl]amino}benzoic acid;
3-{[4-(hydroxymethyl)-2-methoxyphenoxy]methyl}benzoic acid;
5-phenyl[1,2,5]oxadiazolo[3,4-b]pyrazine;
2,3,9-trimethoxy-6a,12a-dihydrochromoeo[3,4-b]chromen-12(6H)-one;
4-[(4-chlorobenzyl)oxy]-3-methoxy-N-(2-methyl-5-[1,3]oxazolo[4,5-b]pyridin-2-ylphenyl)benzamide;
3-(1,3-benzothiazol-2-ylthio)propanoic acid;
3-{[(4-ethoxyphenoxy)acetyl]amino}benzoic acid;
N-[4-chloro-3-(trifluoromethyl)phenyl]-1-[(2,5-dimethoxyphenyl)sulfonyl]piperidine-4-carboxamide;
N-[3,5-bis(trifluoromethyl)phenyl]-2-[(5-nitro-1H-benzimidazol-2-yl)thio]acetamide;
5-[4-(4-tert-butylbenzoyl)piperazin-1-yl]-2-nitro-N-(pyridin-3-ylmethyl)aniline;
3-{[2-chloro-4-(hydroxymethyl)phenoxy]methyl}benzoic acid;
{[(2-chlorophenyl)amino][(3-fluorobenzyl)thio]methylene}malononitrile; and
{[(4-bromophenyl)amino][(4-fluorobenzyl)thio]methylene}malononitrile;
(3,5-di-t-butyl-4-hydroxybenzylidene)malononitrile;
2-amino-N-{5-[6-(dimethylamino)-9H-purin-9-yl]-4-hydroxy-2-(hydroxymethyl)tetrahydrofuran-3-yl}-3-(4-methoxyphenyl)propanamide;
1,3,6-trihydroxy-7-methoxy-2,8-bis(3-methylbut-2-en-1-yl)-9H-xanthen-9-one;
(3S,3aR,4R,6S,6aR,7S,8R,9bR)-6-acetoxy-4-(butyryloxy)-3,3a-dihydroxy-3,6,9-trimethyl-8-{[(2Z)-2-methylbut-2-enoyl]oxy}-2-oxo-2,3,3a,4,5,6,6a,7,8,9b-decahydroazuleno[4,5-b]furan-7-ylhexanoate;
(1E,5S)-6,6,6-trifluoro-5-hydroxy-1,5-diphenylhex-1-en-3-one;
2-(4-methoxyphenyl)-1,3-benzothiazole;
Ethyl-4-amino-2-{[(1E)-3-(4-methylphenyl)-3-oxoprop-1-en-1-yl]thio}pyrimidine-5-carboxylate;
3-[(5-chloro-1,3-benzothiazol-2-yl)thio]-5,5-dimethylcyclohex-2-en-1-one;
(2E)-3-[(5-methyl-1H-benzimidazol-2-yl)thio]-1-phenylprop-2-en-1-one;
11,12-dihydroxy-3-isobutyl-4,5,8-trimethyl-3,3a,4,6a,9,10,11,12-octahydro-1H-cycloundeca[d]isoindole-1,15(2H)-dione;
3-(hydroxymethyl)-6-isopropyl-5-[(2-methylbut-2-enoyl)oxy]-2-oxocyclohex-3-en-1-yl4-hydroxy-2-methylbut-2-enoate;
1,11,12-trihydroxy-2,16-dioxo-11,20-epoxypicras-3-en-15-yl2-hydroxy-2-methylbutanoate;
1-[3,5-bis(trifluoromethyl)phenyl]-3-[3-chloro-4-(isopropylsulfonyl)-2-thienyl]urea;
a pharmaceutically acceptable salt thereof;
and any combination thereof.

13. (canceled)

14. (canceled)

15. (canceled)

16. The pharmaceutical composition of claim 12, wherein the composition further comprises an additional therapeutic agent.

17. A compound selected from:
1-[(1S,3R,5R,7S)-3-(3,4-dimethylphenyl)adamantan-1-yl]methanamine;
5-chloro-7-[(2-chloro-6-fluorophenyl)(pyridin-2-ylamino)methyl]quinolin-8-ol;
(1S,2R,3S,6S)-1-benzoyl-6-tert-butylspiro[2.5]octane-1,2-dicarbonitrile;
N-[(4-chlorophenyl)(2-hydroxy-1-naphthyl)methyl]-3-methoxybenzamide;
Ethyl-1-benzyl-5-[3-(tert-butylamino)-2-hydroxypropoxy]-2-methyl-1H-indole-3-carboxylate;
5-chloro-7-[(2,5-dimethylphenyl)(pyridin-2-ylamino)methyl]quinolin-8-ol;
N-[5-(2,3-dihydro-1,4-benzodioxin-6-yl)-2-methyl-3-furoyl]glycine;
[3-(1,3-benzothiazol-2-yl)phenoxy]acetic acid;
N-(2-fluorophenyl)-2-{[5-(3-methylphenyl)-1,3,4-oxadiazol-2-yl]thio}acetamide;
N-[4-(6-methyl-1,3-benzothiazol-2-yl)phenyl]-2-[(5-methyl-1,3,4-thiadiazol-2-yl)thio]acetamide;
2-(2-bromo-4-tert-butylphenoxy)-N-(2-methoxy-5-nitrophenyl)acetamide;
Methyl-5-{[(4-fluoro-2-methylphenyl)sulfonyl](isonicotinoyl)amino}-2-methyl-1-benzofuran-3-carboxylate;
1-(4-chlorobenzyl)-N-(pyridin-3-ylmethyl)-1H-pyrazolo[3,4-d]pyrimidin-4-amine;
2-{[5-(4-chlorophenyl)-4-(3-methylphenyl)-4H-1,2,4-triazol-3-yl]thio}-N-(3-methoxyphenyl)acetamide;
N-phenyl-3-[(4-phenyl-1,3-thiazol-2-yl)thio]propanamide;
Ethyl-[3-(1,3-benzothiazol-2-yl)phenoxy]acetate;
2-methyl-3-[(4-methylpiperidin-1-yl)carbonyl]naphtho[2,3-b]furan-4,9-dione;
N-(3-phenylpropyl)-10H-phenothiazine-10-carboxamide;
5-(4-chlorophenyl)-2-pyrrolidin-1-yl-6H-1,3,4-thiadiazine;
7-(4-fluorophenyl)-1-(2-furylmethyl)-5,5-bis(trifluoromethyl)-5,8-dihydropyrimido[4,5-d]pyrimidine-2,4(1H,3H)-dione;
3-{[(2-bromo-4-methylphenoxy)acetyl]amino}benzoic acid;
3-{[4-(hydroxymethyl)-2-methoxyphenoxy]methyl}benzoic acid;
5-phenyl[1,2,5]oxadiazolo[3,4-b]pyrazine;
2,3,9-trimethoxy-6a,12a-dihydrochromoeo[3,4-b]chromen-12(6H)-one;
4-[(4-chlorobenzyl)oxy]-3-methoxy-N-(2-methyl-5-[1,3]oxazolo[4,5-b]pyridin-2-ylphenyl)benzamide;
3-(1,3-benzothiazol-2-ylthio)propanoic acid;
3-{[(4-ethoxyphenoxy)acetyl]amino}benzoic acid;
N-[4-chloro-3-(trifluoromethyl)phenyl]-1-[(2,5-dimethoxyphenyl)sulfonyl]piperidine-4-carboxamide;
N-[3,5-bis(trifluoromethyl)phenyl]-2-[(5-nitro-1H-benzimidazol-2-yl)thio]acetamide;
5-[4-(4-tert-butylbenzoyl)piperazin-1-yl]-2-nitro-N-(pyridin-3-ylmethyl)aniline;
3-{[2-chloro-4-(hydroxymethyl)phenoxy]methyl}benzoic acid;
{[(2-chlorophenyl)amino][(3-fluorobenzyl)thio]methylene}malononitrile; and
{[(4-bromophenyl)amino][(4-fluorobenzyl)thio]methylene}malononitrile;
(3,5-di-t-butyl-4-hydroxybenzylidene)malononitrile;
2-amino-N-{5-[6-(dimethylamino)-9H-purin-9-yl]-4-hydroxy-2-(hydroxymethyl)tetrahydrofuran-3-yl}-3-(4-methoxyphenyl)propanamide;
1,3,6-trihydroxy-7-methoxy-2,8-bis(3-methylbut-2-en-1-yl)-9H-xanthen-9-one;
(3S,3aR,4R,6S,6aR,7S,8R,9bR)-6-acetoxy-4-(butyryloxy)-3,3a-dihydroxy-3,6,9-trimethyl-8-{[(2Z)-2-methylbut-2-enoyl]oxy}-2-oxo-2,3,3a,4,5,6,6a,7,8,9b-decahydroazuleno[4,5-b]furan-7-ylhexanoate;
(1E,5S)-6,6,6-trifluoro-5-hydroxy-1,5-diphenylhex-1-en-3-one;
2-(4-methoxyphenyl)-1,3-benzothiazole;
Ethyl-4-amino-2-{[(1E)-3-(4-methylphenyl)-3-oxoprop-1-en-1-yl]thio}pyrimidine-5-carboxylate;
3-[(5-chloro-1,3-benzothiazol-2-yl)thio]-5,5-dimethylcyclohex-2-en-1-one;
(2E)-3-[(5-methyl-1H-benzimidazol-2-yl)thio]-1-phenylprop-2-en-1-one;
11,12-dihydroxy-3-isobutyl-4,5,8-trimethyl-3,3a,4,6a,9,10,11,12-octahydro-1H-cycloundeca[d]isoindole-1,15(2H)-dione;
3-(hydroxymethyl)-6-isopropyl-5-[(2-methylbut-2-enoyl)oxy]-2-oxocyclohex-3-en-1-yl4-hydroxy-2-methylbut-2-enoate;
1,11,12-trihydroxy-2,16-dioxo-11,20-epoxypicras-3-en-15-yl2-hydroxy-2-methylbutanoate;
1-[3,5-bis(trifluoromethyl)phenyl]-3-[3-chloro-4-(isopropylsulfonyl)-2-thienyl]urea;
a pharmaceutically acceptable salt thereof;
and any combination thereof for use in regulating skin pigmentation.

18. (canceled)

19. (canceled)

20. (canceled)

21. An assay for identifying a compound that modulate the expression or activity of Microphthalmia Transcription Factor (MITF), the method comprising:
(i) contacting a cell with a test compound, wherein the cell comprises a reporter gene operably linked to a promoter of a direct transcriptional target of MITF, and wherein the cell is capable of producing MITF; and
(ii) determining the amount of reporter gene expression after incubation with the test compound;
wherein a change in the expression of the reporter gene relative to a control or reference indicates that the compound modulates the expression or activity of MITF.

22. The assay of claim 21, wherein the test compound inhibits the expression or activity of MITF relative to a control or reference.

23. The assay of claim 21, wherein the promoter is a promoter of TRPM1 gene.

24. The assay of claim 21, wherein the reporter gene is luciferase gene.

25. The assay of claim 21, wherein the cell endogenously produces MITF.

26. The assay of claim 21, wherein the cell is a melanocyte or from a melanoma cell line.

27. (canceled)

28. The assay of claim 21, wherein the test compound is tested at 2 or more different concentrations.

29. (canceled)

30. (canceled)

31. The assay of claim 21, wherein the selected compound has a Log P value less than 7.

32. (canceled)

33. (canceled)

34. A compound selected by the assay of claim 21.
